(19) United States
(12) Patent Application Publication
(10) Pub. No.: US 2015/0272480 A1
(43) Pub. Date: Oct. 1, 2015

(54) ACCELERATION SENSOR OUTPUT PROCESSING PROGRAM, PROCESSING METHOD, PROCESSING APPARATUS, AND GAIT ASSESSMENT PROGRAM

(71) Applicant: FUJITSU LIMITED, Kawasaki-shi (JP)

(72) Inventors: Yosuke Senta, Kawasaki (JP); Fumio NAGASHIMA, Ota (JP); Takanao Sugimoto, Kawasaki (JP); Katsuyoshi Yamaguchi, Yokohama (JP); TAKEO KITA, Kawasaki (JP)

(21) Appl. No.: 14/735,936

(22) Filed: Jun. 10, 2015

Related U.S. Application Data
(63) Continuation of application No. PCT/JP2012/082240, filed on Dec. 12, 2012.

(51) Int. Cl. A61B 5/11; A61B 5/00

(52) U.S. Cl. CPC A61B 5/112; A61B 5/1122; A61B 5/725; A61B 5/7278; A61B 5/1123; A61B 2562/0219

(57) ABSTRACT

An acceleration sensor output processing program for causing a computer to execute a process having generating an outer product of a gravity component vector that is extracted from a sensor value of a triaxial acceleration sensor which is detected during walking by a low pass filter and a swing component vector that is extracted from the sensor value by a high pass filter; obtaining a first value corresponding to a magnitude of the outer product; determining an odd-numbered walking period and an even-numbered walking period of the walking; and generating a magnitude of acceleration in one and the other horizontal directions by inverting a sign of the first value in accordance with a result of the determining.

### CROSS-REFERENCE TO RELATED APPLICATION

This application is a continuation application of International Application PCT/JP2012/082240 filed on Dec. 12, 2012 and designated the U.S., the entire contents of which are incorporated herein by reference.

### FIELD

The present invention relates to an acceleration sensor output processing program, a processing method, a processing apparatus, and a gait assessment program.

### BACKGROUND

Recently available mobile devices such as mobile phones include various sensors and are equipped with various functions that utilize sensor output values. For example, a mobile device including an acceleration sensor determines a current state of the mobile device based on sensor values from the acceleration sensor.

For example, mobile devices that utilize sensor values of an acceleration sensor to assess a walking state of a user are proposed. Examples of such mobile devices are disclosed in Patent Literature 1 and 2. Patent Literature 1 and 2 describe counting the number of steps of an owner of a mobile device based on sensor values of an acceleration sensor and obtaining, by arithmetic operations, a magnitude of acceleration in a vertical direction (a gravitational direction or an up-down direction) and a magnitude of acceleration in a horizontal direction while walking from sensor values.

In addition, gait assessment is described in Patent Literature 3 and the like.

### CITATION LIST

### Patent Literature

Patent Literature 1: Japanese Laid-open Patent Publication No. 2010-257395

Patent Literature 2: Japanese Patent Application No. 2010-206797

Patent Literature 3: Japanese Laid-open Patent Publication No. 2011-251013

Patent Literature 4: Japanese Laid-open Patent Publication No. 2010-271167

Patent Literature 5: Japanese Laid-open Patent Publication No. 2008-33526

Patent Literature 6: Japanese Patent No. 4443978

### SUMMARY

However, in order to assess a walking state, a magnitude of acceleration in one and the other horizontal directions or a magnitude of acceleration in one and the other front-back directions is desirably detected in addition to a magnitude of acceleration in a vertical direction. Furthermore, magnitudes of acceleration in a leftward direction and a rightward direction or magnitudes of acceleration in a forward direction and a backward direction are desirably detected.

A sensor value that is output in a coordinate system of an acceleration sensor can be converted to an up-down, left-right, and front-back coordinate system by a computing process utilizing a rotation matrix. However, this computing process requires a computation involving a trigonometric function and is not an easy task for a microprocessor that is only capable of performing a simple computing process such as arithmetic operations. As a result, a microprocessor capable of performing a more sophisticated computing process is required (desired).

One embodiment is a non-transitory computer-readable storage medium storing therein an acceleration sensor output processing program for causing a computer to execute,

a process comprising:
generating an outer product of a gravity component vector that is extracted from a sensor value of a triaxial acceleration sensor which is detected during walking by a low pass filter and a swing component vector that is extracted from the sensor value by a high pass filter;
obtaining a first value corresponding to a magnitude of the outer product;
determining an odd-numbered walking period and an even-numbered walking period of the walking; and
generating a magnitude of acceleration in one and the other horizontal directions by inverting a sign of the first value in accordance with a result of the determining.

The object and advantages of the invention will be realized and attained by means of the elements and combinations particularly pointed out in the claims.

It is to be understood that both the foregoing general description and the following detailed description are exemplary and explanatory and are not restrictive of the invention.

### BRIEF DESCRIPTION OF DRAWINGS

FIG. 1 is a configuration diagram of a mobile device according to the present embodiment.

FIG. 2 is a diagram schematically illustrating movements of the trunk and both legs in human walking.

FIG. 3 is a diagram for describing a sensor coordinate system and a coordinate system of a walker.

FIGS. 4A to 4C are sequence diagrams illustrating a computing process for obtaining a magnitude of acceleration in a vertical direction of a swing of a walker from a sensor output of an acceleration sensor when a posture of the acceleration sensor is unknown.

FIGS. 5A to 5C are sequence diagrams illustrating a computing process for obtaining acceleration in a horizontal direction of a swing of a walker from a sensor output of an acceleration sensor when a posture of the acceleration sensor is unknown.

FIG. 6 is a diagram illustrating a relationship among the swing component S that accompanies walking, a component h of S in the horizontal direction H, and a component f of S in the front-back direction F.

FIG. 7 is a diagram illustrating a processing function of the acceleration sensor output processing program 15 in the sub microprocessor 14 according to the first embodiment.

FIG. 8 is a sequence diagram illustrating a computing process for obtaining acceleration in one and the other horizontal directions in the acceleration sensor output processing program according to the first example of the first embodiment.

FIG. 9 is a diagram illustrating an example of an output |H| of the absolute value computation processing unit 32 and an output h of the multiplier MX in the computing process illustrated in FIG. 8.

FIG. 10 is a diagram for explaining a method of generating the coefficients +1 and −1 illustrated in FIG. 9.

FIG. 11 is a sequence diagram illustrating a computing process for obtaining acceleration in one and the other horizontal directions in the acceleration sensor output processing program according to the first example of the first embodiment.

FIG. 12 is a diagram for explaining a case where a change of the magnitude |v| of the vertical movement and the magnitude |H| of a movement in the horizontal direction illustrated in FIG. 10 are out of phase.

FIGS. 13A to 13C are diagrams for explaining a relationship between a vector of the outer product S×G and an arbitrary vector K.

FIG. 14 is a sequence diagram illustrating a computing process for obtaining acceleration in one and the other horizontal directions in the acceleration sensor output processing program according to the second example of the first embodiment.

FIG. 15 is a sequence diagram illustrating a computing process for obtaining acceleration in one and the other horizontal directions in the acceleration sensor output processing program according to the third example of the first embodiment.

FIG. 16 is a diagram explaining the fourth example of the first embodiment.

FIG. 17 is a sequence diagram illustrating a computing process for obtaining acceleration in one and the other horizontal directions in the acceleration sensor output processing program according to the fourth example of the first embodiment.

FIG. 18 is a diagram that explains a first example of a gait assessment program.

FIG. 19 is a flow chart for obtaining maximum acceleration by a gait assessment program.

FIG. 20 is a flow chart of a gait assessment according to a gait assessment program that utilizes an acceleration ratio.

FIG. 21 is a diagram illustrating an example of a transition over time of the ratio obtained in FIG. 20.

FIG. 22 is a diagram that explains a second example of a gait assessment program.

FIG. 23 is a flow chart for extracting areas Peng and Neng of squares of acceleration according to the second example.

FIG. 24 is a diagram that explains a third example of a gait assessment program.

FIG. 25 is a diagram that explains a fourth example of a gait assessment program.

FIG. 26 is a diagram illustrating a function of an acceleration sensor output processing program according to the second embodiment.

FIG. 27 is a diagram illustrating a relationship between acceleration Hf in the horizontal direction and acceleration Vf in the vertical direction which have been separated by band pass filters according to the principles described above.

FIG. 28 is a sequence diagram illustrating a computing process for obtaining a magnitude of acceleration in one and the other horizontal directions and a magnitude of acceleration in one and the other front-back directions in the acceleration sensor output processing program according to the second embodiment.

FIG. 29 is a sequence diagram illustrating a computing process for obtaining acceleration in one and the other horizontal directions and acceleration in one and the other front-back directions in the acceleration sensor output processing program according to the second embodiment.

FIG. 30 is a diagram illustrating a function of an acceleration sensor output processing program according to the third embodiment.

FIG. 31 is a diagram illustrating a relationship between a vertical movement and a front-back movement in walking.

FIG. 32 is a sequence diagram illustrating a computing process for obtaining a magnitude of acceleration in the horizontal direction and a magnitude of acceleration in the front-back direction in the acceleration sensor output processing program according to the third embodiment.

FIG. 33 is a sequence diagram of a computing process by the low pass filter LPF.

FIG. 34 is a sequence diagram of a computing process by the high pass filter HPF.

FIG. 35 is a sequence diagram of a computing process by the band pass filter BPF.

### DESCRIPTION OF EMBODIMENTS

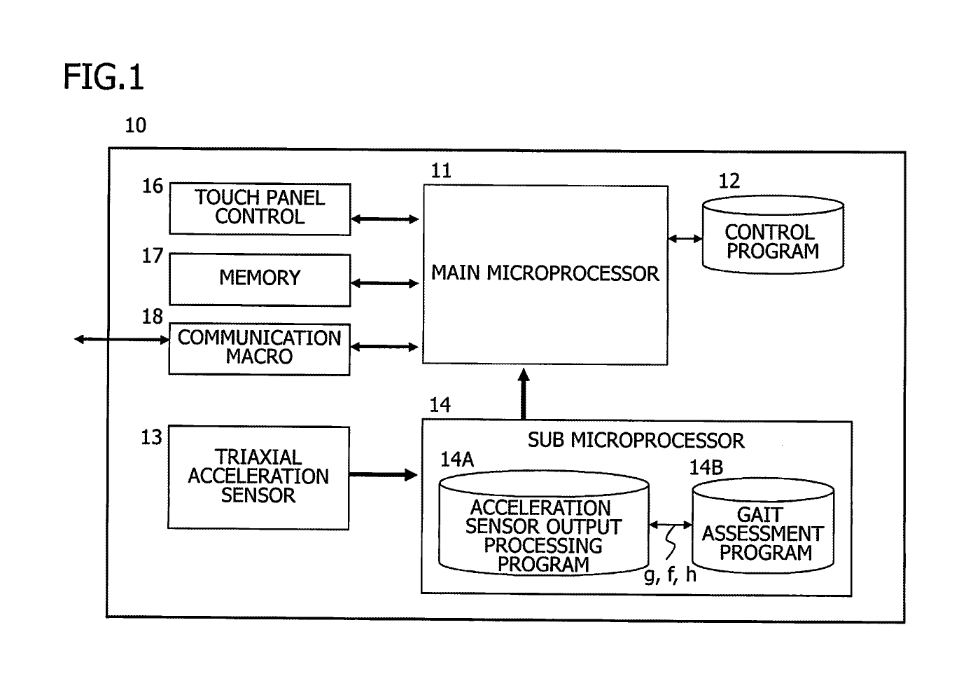

FIG. 1 is a configuration diagram of a mobile device according to the present embodiment. A mobile device 10 that is a smartphone or the like includes a main microprocessor 11, a sub microprocessor 14, and a memory 17. The mobile device 10 further includes, as peripheral resources, a touch panel control unit (circuit) 16 of a display panel (not illustrated), a communication macro circuit 18 that performs wireless communication or the like, and the like. Furthermore, the mobile device 10 includes a triaxial acceleration sensor 13.

The main microprocessor 11 executes a control program 12 to realize various functions that are included in the mobile device. The sub microprocessor 14 executes an acceleration sensor output processing program 14A and performs a computing process on a sensor output that is output in a sensor coordinate system of the acceleration sensor 13 to output acceleration g in a vertical direction, acceleration h in one and the other horizontal directions or in the horizontal direction, and acceleration f in one and the other front-back directions or in the front-back direction. Furthermore, the sub microprocessor 14 executes a gait assessment program 14B and generates and stores a result of an assessment of a walking state of an owner.

The main microprocessor 11 is capable of a high-speed computing process but consumes a large amount of power. Since it is not desirable to keep the main microprocessor 11 in a constantly operating state, certain restrictions are applied to an operation time of the main microprocessor 11 including temporarily controlling the main microprocessor 11 to an idle state. On the other hand, assessing a walking state requires (includes) constantly acquiring daily walking data and assessing a walking state based on the walking data.

To this end, the sub microprocessor 14 is placed in an active state at least during walking, in which the sub microprocessor 14 receives an input of a sensor value of the acceleration sensor 13 and performs a computing process of a sensor value in a coordinate system of the sensor to generate up-down, left-right, and front-back acceleration data described earlier. However, while the sub microprocessor 14 is only capable of performing a simple computing process such as basic four arithmetic operations and processing of access to the memory, since the sub microprocessor 14 only consumes a small amount of power, the acceleration sensor output processing program 14A may be executed for a long period of time. Furthermore, the sub microprocessor 14 executes the gait assessment program 14B to assess a walking state and store a result of the assessment.

In addition, the main microprocessor 11 is intermittently placed in an operating state and, based on a result of an assessment of a walking state that is generated constantly or over a long period of time by the sub microprocessor 14, converts the assessment value into a value that is readily understood by a user or creates statistical information, and outputs the value or the information to the user.

As described above, the acceleration sensor output processing program 14A that is executed by the sub microprocessor 14 converts a sensor output of the sensor coordinate system into acceleration in a coordinate system of the owner carrying the mobile device by a simple computing process. The simple computing process includes a computing process for obtaining an inner product and an outer product.

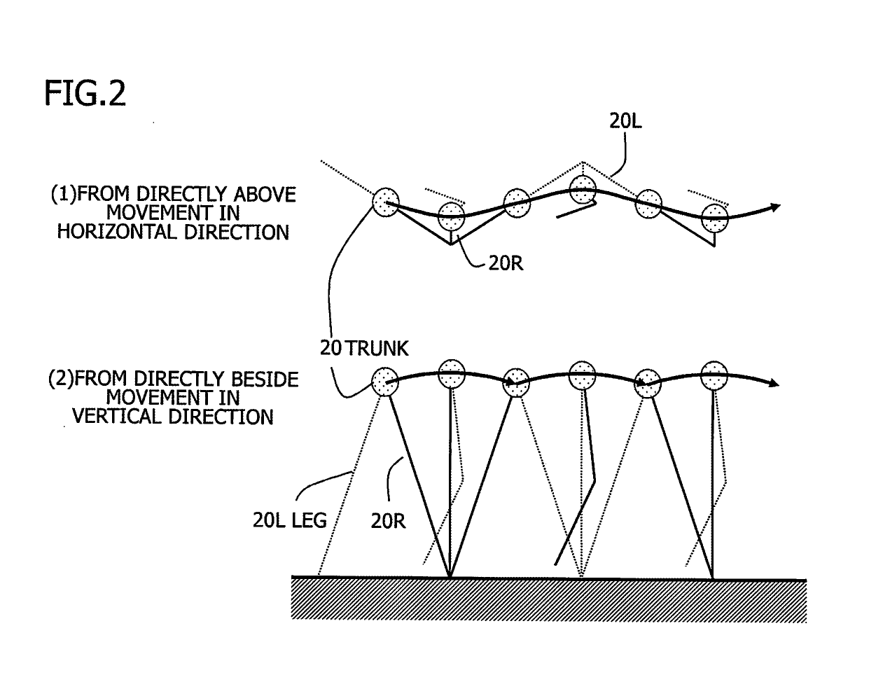

FIG. 2 is a diagram schematically illustrating movements of the trunk and both legs in human walking. A trunk 20 corresponds to a portion from a hip joint to a head while legs 20L and 20R correspond to the left leg and the right leg of a human being. During walking, a human being alternately places the left and right legs 20L and 20R on the ground and swings the trunk 20 up and down, left and right, and back and forth. As illustrated in FIG. 2, a period of vertical movement of the trunk 20 is consistent with a period in which the left and right legs 20L and 20R are placed on the ground. On the other hand, a period of a horizontal swing of the trunk 20 includes swinging toward the left when the left leg 20L is placed on the ground and swinging toward the right when the right leg 20R is placed on the ground. Therefore, the period of a horizontal swing is twice as long (½ frequency) as the period of vertical movement.

In addition, with a human being, during walking, acceleration in a downward direction is greatest at instants where the left and right legs 20L and 20R land on the ground, acceleration in an upward direction is generated when the trunk 20 ascends after landing, and the acceleration in the upward direction is minimized and is switched to the acceleration in the downward direction at a highest point. Furthermore, with acceleration in the horizontal direction, acceleration in the leftward direction is generated when the left leg 20L lands and acceleration in the rightward direction is generated when the right leg 20R lands.

In the present embodiment, such feature points of human walking are utilized to convert a sensor output of a sensor coordinate system (X, Y, Z) into a coordinate system (up and down (z axis), left and right (x axis), front and back (y axis)) of a walker by arithmetic operations.

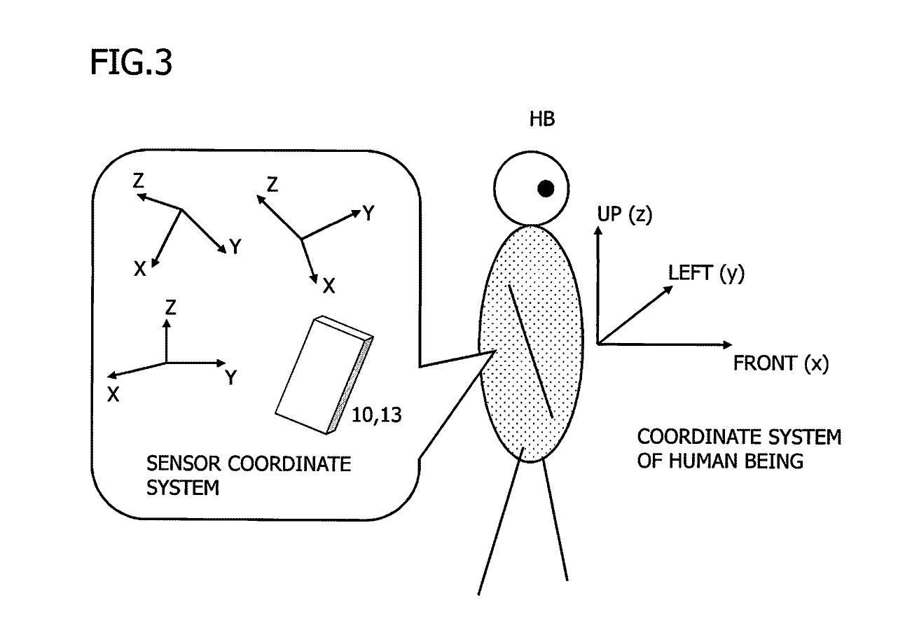

FIG. 3 is a diagram for describing a sensor coordinate system and a coordinate system of a walker. A walker HB carries the mobile device 10 that includes the acceleration sensor 13. Since the mobile device 10 is carried in various directions, a coordinate system of the acceleration sensor 13 is also oriented in various directions. In addition, the acceleration sensor 13 outputs an acceleration vector that is detected in the sensor coordinate system (X, Y, Z). At this point, the acceleration sensor output processing program 15 of the sub microprocessor 14 computes accelerations in a vertical direction, a horizontal direction, and a front-back direction in a coordinate system (x, y, z) of the walker HB from the acceleration vector in the sensor coordinate system. Furthermore, the computing process only includes a simple computing process that is comparable to basic four arithmetic operations. Specifically, the computing process includes computations for obtaining an inner product and an outer product, in which case inner product and outer product computations are performed by arithmetic operations.

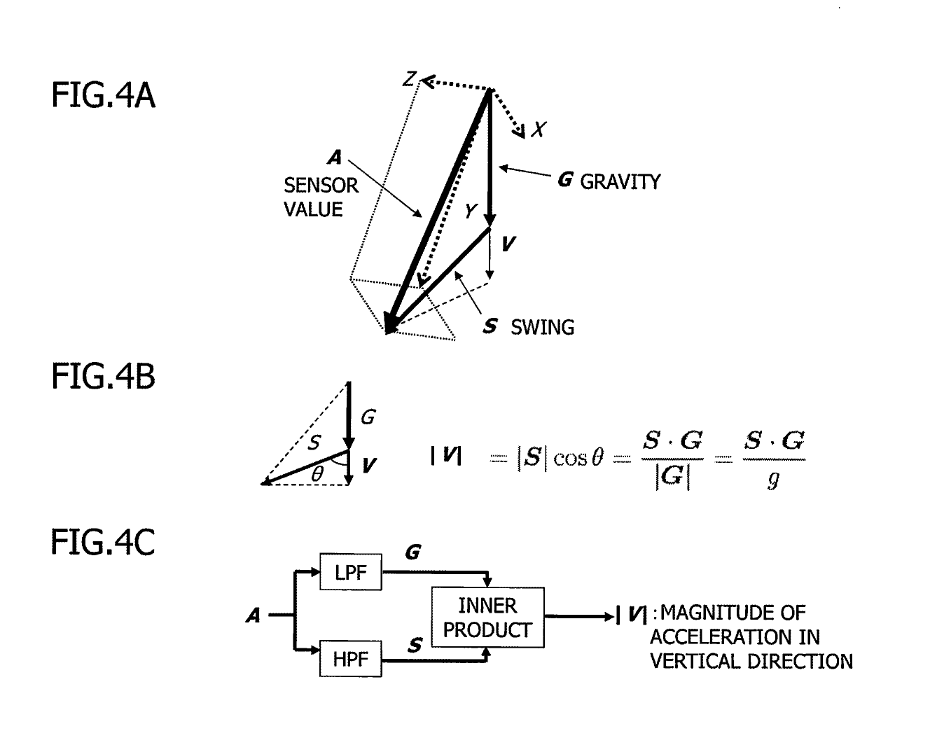

FIGS. 4A to 4C are sequence diagrams illustrating a computing process for obtaining a magnitude of acceleration in a vertical direction of a swing of a walker from a sensor output of an acceleration sensor when a posture of the acceleration sensor is unknown. FIG. 4A illustrates a sensor output A of the acceleration sensor, gravitational acceleration G, and acceleration S of a swing of the walker. The acceleration sensor outputs a coordinate value (X1, Y1, Z1)^{T} in the sensor coordinate system X, Y, Z as the sensor output A. The coordinate value of the sensor output A represents a vector including a direction and a magnitude (scalar value) of the sensor output A.

The sensor value A=(X1, Y1, Z1)^{T} is a composite of a gravitational acceleration vector G and an acceleration vector S of a swing caused by walking. As described earlier with reference to FIG. 2, the swing S caused by walking is a repetitive movement with a given period. Therefore, by passing the sensor output A through a low pass filter and a high pass filter with appropriate cutoff frequencies, the sensor output A can be separated into swing acceleration S and gravitational acceleration G.

As illustrated in FIG. 4B, acceleration in the vertical direction that accompanies the swing of walking is a component V that is parallel to gravity G of the swing S. If θ denotes an angle formed by S and G, then a magnitude |V| of V may be expressed as

|V|=|S| cos θ (Equation 1).

Let us now consider an inner product (dot product) of S and G. Since an inner product is obtained by multiplying the X, Y, and Z components of the vectors by each other and then adding up the products, even the sub microprocessor 14 with limited processing power can readily calculate an inner product. Specifically, if S=(aₓ, a_{y}, a_{z}) and G=(bᵣ, b_{y}, b_{z}), then an inner product can be obtained by the following arithmetic expression.

S·G=aₓbₓ+a_{y}b_{y}+aₓb_{y}

By definition, the inner product of S and G is expressed as

S·G=|S∥G| cos θ (Equation 2).

Applying Equation 2 to Equation 1 above results in

|V|=|S| cos θ=|S∥G| cos θ/|G|=S·G/|G| (Equation 3).

Since G denotes gravitational acceleration, a magnitude |G| thereof is approximately constant all over the world. Specifically, as shown in FIG. 4C, by passing the sensor value A through a low pass filter LPF and a high pass filter HPF and computing an inner product of respective outputs, a numerical value that is proportional to the magnitude |V| of the acceleration V in the vertical direction accompanying a walking motion can be obtained. In other words, while the acceleration V has positive and negative signs, due to |V| in Equation (3), at least a magnitude of acceleration in an upward direction and a magnitude of acceleration in a downward direction can be obtained.

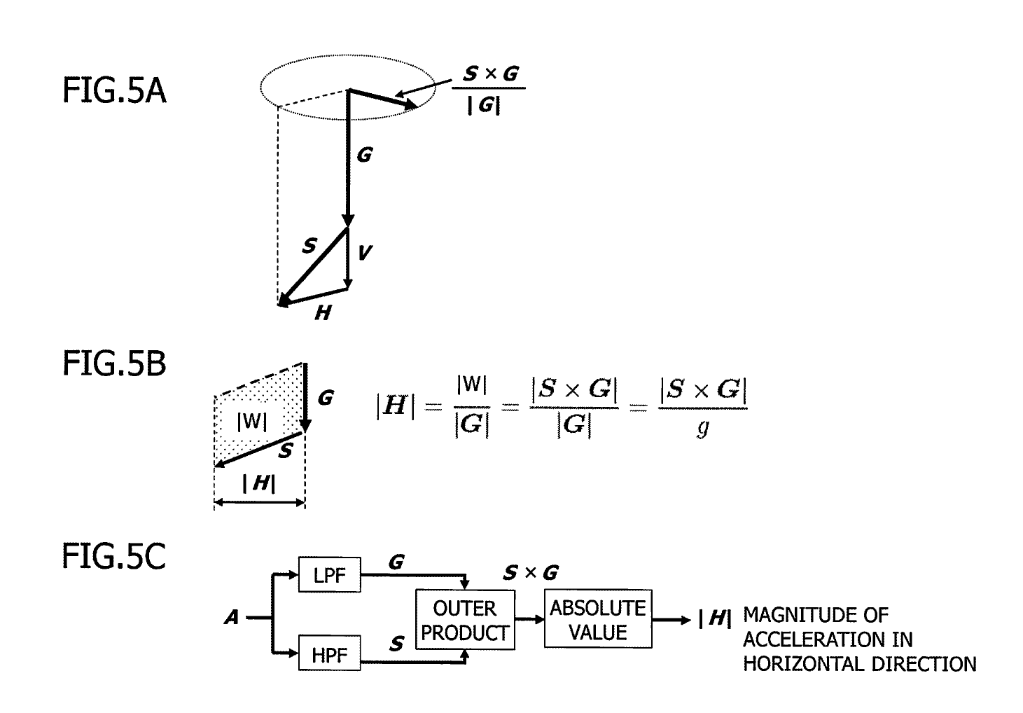

FIGS. 5A to 5C are sequence diagrams illustrating a computing process for obtaining acceleration in a horizontal direction of a swing of a walker from a sensor output of an acceleration sensor when a posture of the acceleration sensor is unknown. In a similar manner to FIGS. 4A to 4C, as illustrated in FIG. 5A, the sensor output A of the acceleration sensor is considered to be a composite of acceleration S of a swing caused by walking and gravitational acceleration G.

When a swing component S is considered to be a sum of a component V that is parallel to G and a component H that is perpendicular to G, then H is considered to be a horizontal component of a swing that accompanies a walking state. Let us now consider an outer product of S and G. Since an outer product is also obtained by multiplication and subtraction of elements of the respective vectors, even a sub microprocessor with limited processing power can readily calculate an outer product. Specifically, if S=(aₓ, a_{y}, a_{z}) and G=(bᵣ, b_{y}, b_{z}), then an outer product is obtained by the following arithmetic expression.

S×G=(a_{y}b_{z}−a_{z}b_{y},a_{z}bₓ−aₓb_{z},aₓb_{y}−a_{y}bₓ)

By definition, the outer product of S and G is a vector W whose direction is perpendicular to both S and G and whose magnitude is equal to an area |W| of a parallelogram that is formed by S and G. In other words,

|W|=|S×G| (Equation 4).

As shown in FIG. 5B, the area |W| of the parallelogram can also be described as |W|=|G∥H|. Applying this equation |W|=|G∥H| to Equation 4 results in |H|=|W|/|G|=|S×G|/|G|.

Considering that |G| is approximately constant all over the world in a similar manner to FIGS. 4A to 4C, as shown in FIG. 5C, by extracting gravity direction acceleration G and acceleration S of a swing by passing the sensor output A through the low pass filter LPF and the high pass filter HPF, computing an outer product G×S thereof, obtaining an absolute value of the outer product, and dividing the absolute value by |G|, a numerical value that is proportional to a magnitude |H| of the horizontal component H of the swing S accompanying a walking state can be obtained.

Moreover, there are two points to keep in mind when using this method. One is a method of obtaining an absolute value of outer product. While obtaining an absolute value requires (involves) squaring and adding up the respective elements of a vector of the outer product S×G and obtaining a square root of the sum value, this requires (uses) calculation cost. Therefore, in FIGS. 5A to 5C, when only performing a magnitude comparison of a magnitude of a horizontal component, a result itself of respective squared elements of the outer product vector can be used without obtaining a square root. In other words, even with the sub microprocessor 14, a magnitude (|H|²) corresponding to the magnitude |H| of acceleration in the horizontal direction can be obtained by computation. Therefore, the computation for obtaining the absolute value in FIGS. 5A to 5C may be a computation including adding up squares of the respective elements of a vector of the outer product S×G and dividing the result by |G|.

The second point to keep in mind is that, since an absolute value is used, an obtained numeral value is always positive. In other words, the obtained result is |H| and not a signed value such as h=|S| sin θ. However, when only performing a magnitude comparison of a magnitude of a horizontal component, a signed value that enables a distinction to be made between horizontal directions is not necessary.

[Outline of Present Embodiment]

According to the method illustrated in FIGS. 4A to 4C described above, upward and downward swing component is separated. In addition, according to the method illustrated in FIGS. 5A to 5C, swing component in the horizontal direction is separated. However, the swing component S cannot be separated into a component in the leftward direction and the rightward direction and a component in the forward direction and the backward direction.

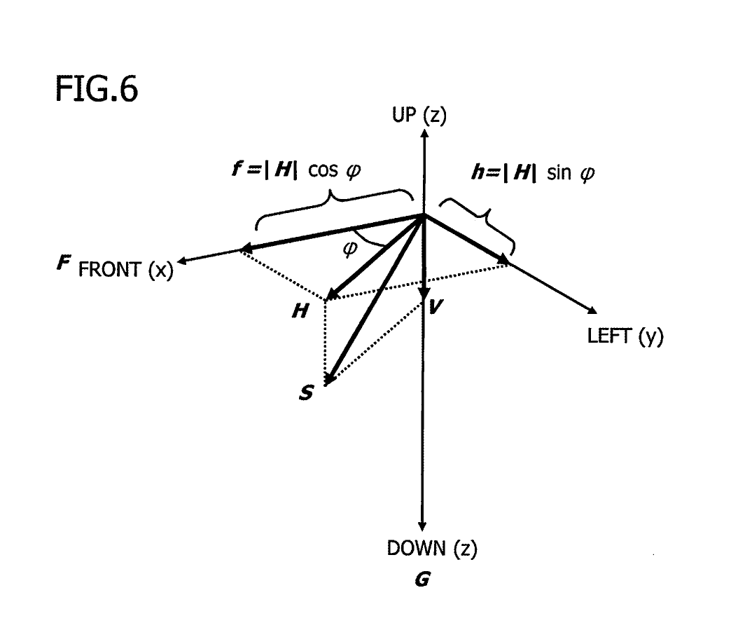

FIG. 6 is a diagram illustrating a relationship among the swing component S that accompanies walking, a component h of S in the horizontal direction H, and a component f of S in the front-back direction F. A vertical axis z in a direction of gravity of a walker, a front-back axis x, and a horizontal axis y are illustrated in FIG. 6. The swing component S that accompanies walking is a composite of a gravity direction component V and a horizontal direction component H. In addition, if an angle formed between the horizontal direction component H and a forward direction F (the front-back axis x) is denoted by φ, then a magnitude f of acceleration in the forward direction may be expressed as f=|H| cos φ and a magnitude h of acceleration in the leftward direction may be expressed as h=|H| sin φ. Since these magnitudes are respectively signed, f=|H| cos φ signifies a magnitude of acceleration in the front-back direction and h=|H|sin φ signifies a magnitude of acceleration in the horizontal direction.

Therefore, in the present embodiment, by taking feature points of a walking motion into consideration, with respect to the swing S that accompanies walking of a walker, an angle φ formed between the forward direction F and a swing H of a horizontal component is computed to obtain a magnitude |H| cos φ in the front-back direction and a magnitude |H| sin φ in the horizontal direction. However, first, although a distinction between left and right is not made, a first embodiment in which acceleration in one and the other horizontal directions is obtained will be described. Subsequently, a second embodiment in which acceleration in one and the other front-back directions is obtained in addition to the acceleration in one and the other horizontal directions, and a third embodiment in which acceleration that enables a distinction to be made between left and rightward directions and acceleration that enables a distinction to be made between forward and backward directions will be described.

### First Example of First Embodiment

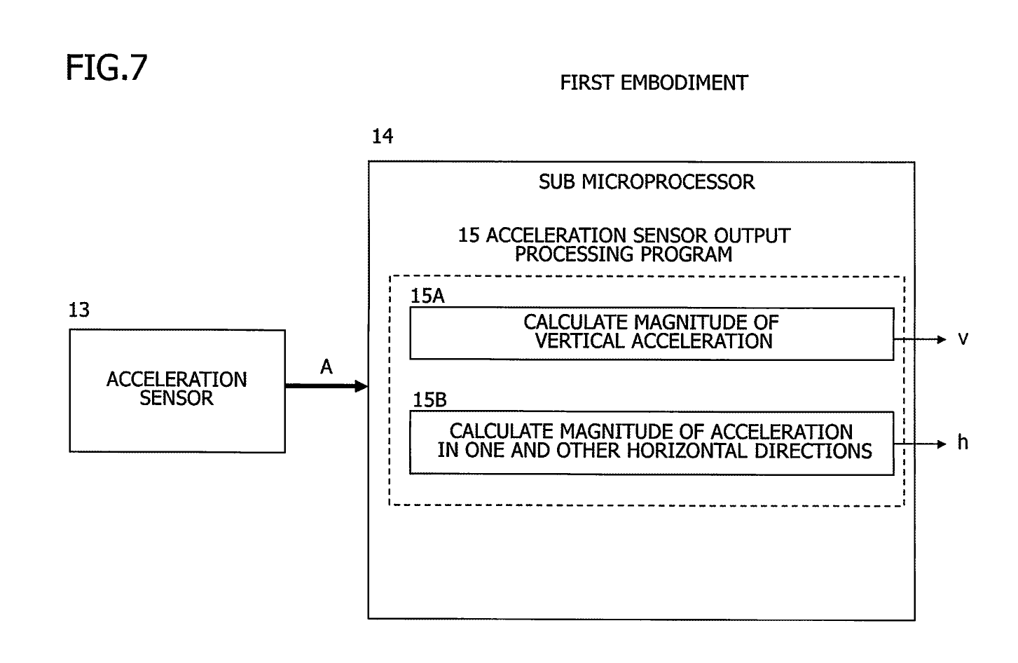

FIG. 7 is a diagram illustrating a processing function of the acceleration sensor output processing program 15 in the sub microprocessor 14 according to the first embodiment. The acceleration sensor output processing program 15 calculates a magnitude of acceleration in the vertical direction (15A) and calculates a magnitude of acceleration in one and the other horizontal directions (15B) from a sensor output A of a sensor coordinate system which is output by the acceleration sensor 13. However, in this example, a magnitude of acceleration that enables a distinction to be made between rightward and leftward directions is not calculated.

As a premise, a movement of the trunk 20 in human walking illustrated in FIG. 2 will be considered. In this case, it is assumed that walking proceeds at a constant speed in a steady state. Since the walker does not bend knees of the supporting legs 20L and 20R, a movement in the vertical direction is generated. Since lengths of the supporting legs 20L and 20R do not change while in contact with the ground, the trunk 20 traces a trajectory of an arc centered on the ankles. In addition, as far as the horizontal direction is concerned, since a center of gravity (more accurately a ZMP (zero moment point)) must be (is) positioned within the soles of the supporting legs 20L and 20R, the trunk 20 swings left and right. In other words, while each supporting leg makes one period in the vertical direction, in the horizontal direction, each supporting leg makes one half period or in other words, the left and right supporting legs combine to make one period.

On the other hand, regarding the front-back direction, an observation of a large number of pieces of actually measured data reveals that, in a steady state other than when starting walking or stopping, forward progress occurs at a constant speed and acceleration in the front-back direction is hardly generated. This is obvious from (demonstrated by) the fact that the upper body moves at a constant speed. Assuming that acceleration is not generated in the front-back direction, an angle φ of the horizontal direction component H in FIG. 6 conceivably only takes values of 90° or −90°.

In consideration thereof, in the first embodiment, whether the angle φ is either +90° or −90° is determined from output information A of the acceleration sensor. Once the angle φ is determined to be either 90° or −90°, acceleration h of one and the other horizontal directions is obtained by multiplying |H| as determined by the computation illustrated in FIGS. 5A to 5C by a coefficient of +1 or −1 that corresponds to the angle φ being either 90° or −90°.

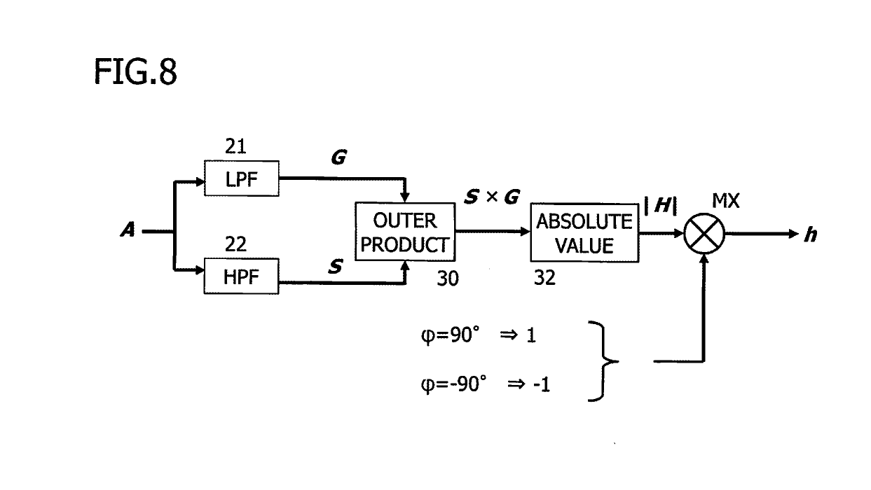

FIG. 8 is a sequence diagram illustrating a computing process for obtaining acceleration in one and the other horizontal directions in the acceleration sensor output processing program according to the first example of the first embodiment. In the computing process illustrated in FIG. 8, an absolute value (or a square of an absolute value) of an outer product S×G that is obtained in a similar manner to FIGS. 5A to 5C are divided by |G| and then multiplied by a coefficient of +1 or −1 corresponding to the angle φ being either 90° or −90°. Specifically, the computing process illustrated in FIG. 8 includes an LPF 21 that extracts a gravity component G and an HPF 22 that extracts a swing component S from an acceleration sensor output A, an outer product computation processing unit 30 that computes an outer product S×G thereof, an absolute value computation processing unit 32 that divides an absolute value (or a square of an absolute value) of the outer product S×G by |G|, and a multiplier MX which multiplies an output |H| (or |H|²) of the absolute value computation processing unit 32 by a coefficient of +1 or −1 corresponding to either 90° or −90° and which outputs one and the other left and right magnitudes h (signed) that take a positive value in one horizontal direction and takes a negative value in the other horizontal direction.

Moreover, as described earlier, a square root may or may not be used in the computation for obtaining an absolute value. Therefore, while a magnitude of acceleration will be hereinafter denoted by |H|, |H|² that is not a square root may be used instead.

The computing process illustrated in FIG. 8 includes the following steps. Specifically, the computing process includes the steps of: generating an outer product S×G of a gravity component vector G extracted by a low pass filter 21 and a swing component vector S extracted by a high pass filter 22 from a sensor value A; obtaining a first value |H| corresponding to a magnitude of the outer product S×G; determining an odd-numbered walking period and an even-numbered walking period of walking (determining whether φ is 90° or −90°); and depending on the determination result, inverting a sign of the first value |H| to generate an acceleration h in one and the other horizontal directions (right and left).

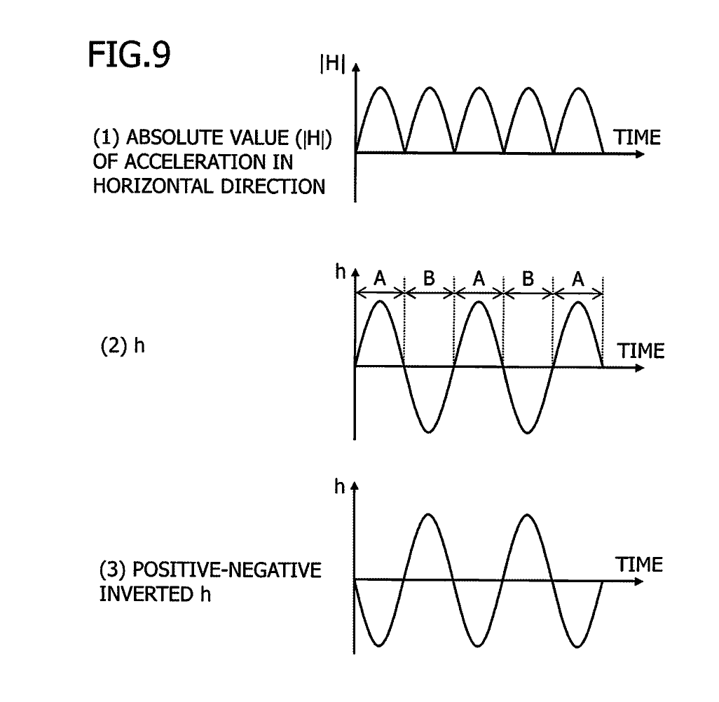

FIG. 9 is a diagram illustrating an example of an output |H| of the absolute value computation processing unit 32 and an output h of the multiplier MX in the computing process illustrated in FIG. 8. The output |H| of the absolute value computation processing unit 32 illustrated in (1) of FIG. 9 does not include a positive or negative sign and simply represents a magnitude. By multiplying the output |H| by coefficients +1 and −1 at appropriate timings A and B to invert signs, the output h that indicates one and the other of left and right by a sign is obtained as illustrated in (2) of FIG. 9. In this case, since a distinction between left and right need not be made (is not made), when the timings of multiplying the coefficients +1 and −1 are reversed to B and A, as illustrated in (3) of FIG. 9, the output h becomes a magnitude of an opposite phase with respect to (2) of FIG. 9.

As described above, in the first embodiment, a magnitude of acceleration when one of the left and right legs of a walker is a supporting leg and a magnitude of acceleration when the other of the left and right legs of a walker is a supporting leg are detected while being distinguished from one another. Therefore, the first embodiment is applied when there is no need to distinguish whether a current supporting leg is the right leg or the left leg when performing gait assessment.

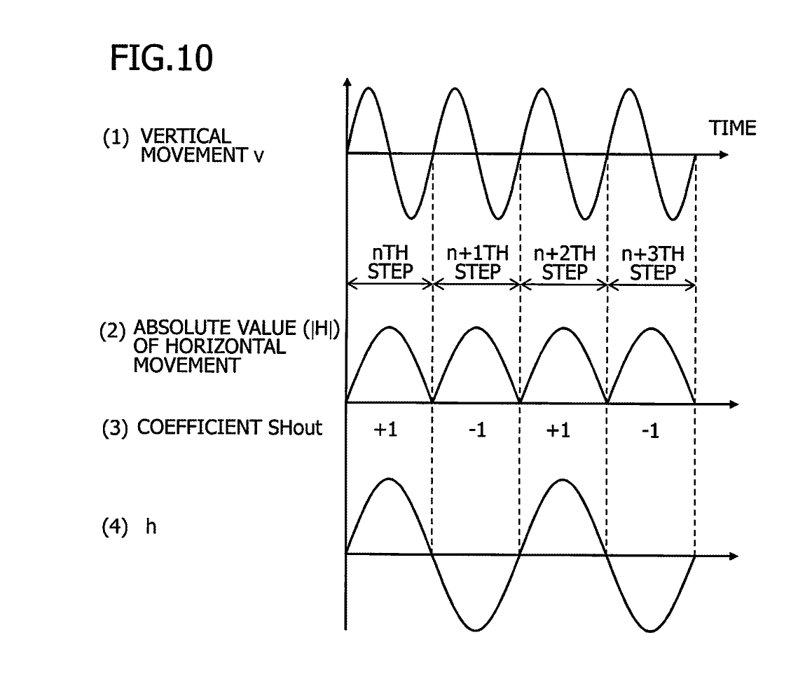

FIG. 10 is a diagram for explaining a method of generating the coefficients +1 and −1 illustrated in FIG. 9. In the first embodiment, the coefficients +1 and −1 are used with the number of steps of walking. Specifically, a walker walks by alternately bringing the right leg and the left leg into contact with the ground and, synchronously, swings in the vertical direction to generate acceleration in the vertical direction. Therefore, signs of the magnitude |v| of the acceleration in the vertical direction described in FIGS. 4A to 4C are counted to detect switching timings of the left and right legs. In other words, when the number of steps indicates an even-numbered step, the coefficient +1 is used on the right leg to output h=+|H|, and when the number of steps indicates an odd-numbered step, the coefficient −1 is used on the left leg to output h=−|H|.

As illustrated in (1) of FIG. 10, the magnitude |v| of acceleration v generated by a vertical movement repetitively takes positive and negative magnitudes for each number of steps. h=+|H| or −|H| illustrated in (3) of FIG. 10 can be generated by detecting switching of the number of steps from a change in the sign of the magnitude |v| of acceleration in the vertical direction and multiplying the magnitude |H| of one and the other horizontal directions illustrated in (2) of FIG. 10 by the coefficient −1 at the n+1th step and the n+3th step (where n denotes an even number) among odd-numbered steps.

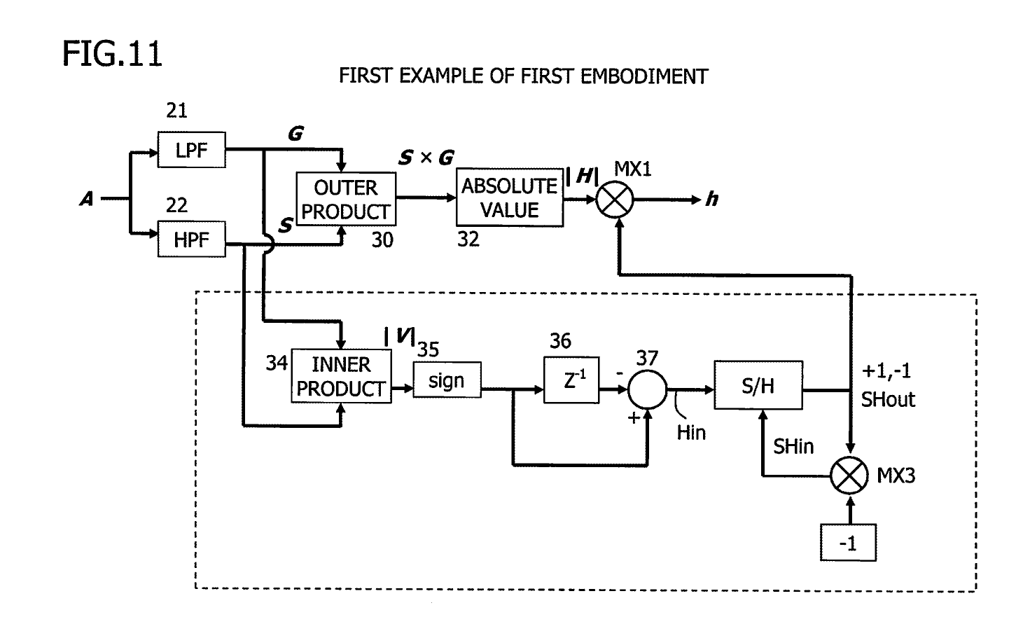

FIG. 11 is a sequence diagram illustrating a computing process for obtaining acceleration in one and the other horizontal directions in the acceleration sensor output processing program according to the first example of the first embodiment. A computing process 33 that generates the coefficients +1 and −1 has been added to the computing process illustrated in FIG. 8. Therefore, in FIG. 11, the LPF 21, the HPF 22, the outer product computation processing unit 30, the absolute value computation processing unit 32, and the multiplier MX1 are the same as those in FIG. 8.

The computing process 33 that generates the coefficients +1 and −1 includes: an inner product computation processing unit 34 that computes an inner product of the gravity component G and the swing component S extracted from the acceleration sensor output A; a sign extracting unit 35 that extracts a sign of an output |v| of the inner product computation processing unit 34; a delay processing unit 36 that delays a sampling timing of sensor output; a subtractor 37; a sample-and-hold processing unit S/H that generates an output SHout of the coefficients +1 and −1; and a multiplier MX3 which multiplies the coefficient SHout by −1 to generate an input SHin to the sample-and-hold processing unit S/H.

As illustrated in (1) of FIG. 10, the sign of the output |v| of the inner product alternately changes between positive and negative. In consideration thereof, a hold input Hin that takes a value of 2 at a timing of a zero-cross point from negative to positive is generated by the delay processing unit 36 and the subtractor 37. −2 is output by the subtractor 37 at a timing of a zero-cross point from positive to negative. Each time the hold input Hin takes a value of 2, the sample-and-hold processing unit S/H holds the input SHin that is expressed as output SHout of multiplier MX3×(−1) and maintains the value of output SHout. Accordingly, every time the vertical movement |v| illustrated in FIG. 10 passes zero-cross points where negative changes to positive, the coefficient (SHout) that is output by S/H and input to the multiplier MX1 alternately changes between +1 and −1.

In addition, by multiplying an absolute value |H| of the outer product S×G by the coefficient SHout (+1, −1), the multiplier MX1 generates a magnitude h of signed acceleration corresponding to one and the other horizontal directions.

The computing process of the first example illustrated in FIG. 10 generates an inner product of a gravity component vector G and a swing component vector S and determines an odd-numbered walking period and an even-numbered walking period of walking, based on an odd-numbered period and an even-numbered period of the inner product, in the determination step (the computation processing unit 33) in the computing process illustrated in FIG. 8 described earlier.

The computing process described above is executed when the sub microprocessor 14 executes the sensor output processing program 15.

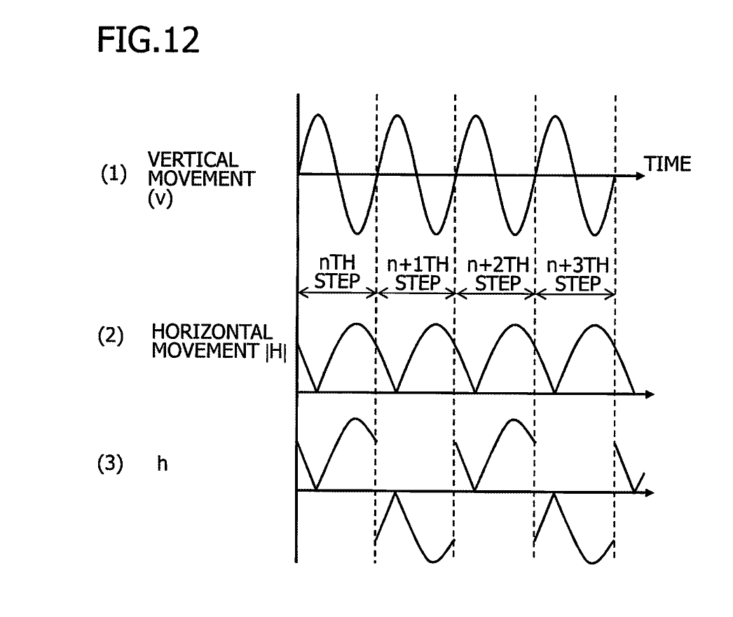

FIG. 12 is a diagram for explaining a case where a change of the magnitude |v| of the vertical movement and the magnitude |H| of a movement in the horizontal direction illustrated in FIG. 10 are out of phase. Depending on a walking form of a walker, there may be cases where a vertical movement and a horizontal movement do not proceed at a fixed phase difference due to bending of the body or the like. Therefore, when the movements go out of phase as illustrated in FIG. 12, changes of a magnitude of acceleration in one and the other of left and right which is illustrated in the sequence diagram in FIG. 11 may occur as illustrated in (3) of FIG. 12.

### Second Example of First Embodiment

In the second example of the first embodiment, the computing process 33 for generating the coefficients +1 and −1 illustrated in FIG. 11 is replaced with a computing process in which the coefficients +1 and −1 are generated according to a sign of an inner product of the outer product S×G of the swing acceleration S and the gravitational acceleration G and an arbitrary vector K.

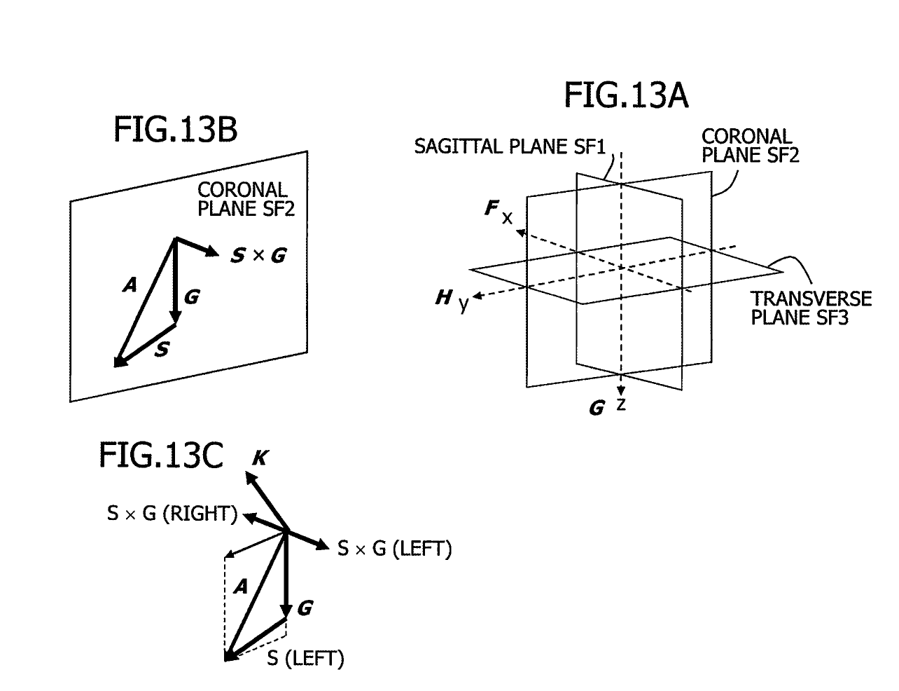

FIGS. 13A to 13C are diagrams for explaining a relationship between a vector of the outer product S×G and an arbitrary vector K. First, a sagittal plane, a coronal plane, and a transverse plane will be described. As illustrated in FIG. 13A, a sagittal plane SF1 is a plane including a front-back axis x and a vertical axis z, a coronal plane SF2 is a plane including a horizontal axis y and the vertical axis z, and a transverse plane SF3 is a plane including the front-back axis x and the horizontal axis y.

In addition, as described earlier, an absolute value of the outer product S×G of the swing acceleration S and the gravitational acceleration G corresponds to a magnitude of acceleration in the horizontal direction. Furthermore, as described earlier, assuming that there is no acceleration in the front-back direction which accompanies walking, the swing acceleration S is always inside the coronal plane SF2.

Therefore, as illustrated in FIG. 13B, the vector of the outer product S×G is a vector that is pointed forward which is a positive direction of the front-back axis x or pointed backward which is a negative direction of the front-back axis x, the front-back axis x being a line of intersection of the sagittal plane SF1 and the transverse plane SF3. In the example illustrated in FIGS. 13A to 13C, since the swing acceleration S is oriented in a leftward direction, the outer product S×G points backward (the negative direction of the front-back axis x). Although not illustrated in FIGS. 13A to 13C, if the swing acceleration S is oriented in a rightward direction, the outer product S×G is to point forward. Utilizing this principle, a determination is made that the swing acceleration S is in a rightward direction if the vector of the outer product S×G is oriented forward and that the swing acceleration S is in a leftward direction if the vector of the outer product S×G is oriented backward.

In the second example of the first embodiment, the coefficients +1 and −1 described earlier are generated utilizing a result of this determination. Moreover, a relationship between the direction of the swing acceleration S and the outer product described above is reversed when G×S is used as the outer product.

Next, a method of determining switching of orientations of the vector of the outer product S×G will be described. As illustrated in FIG. 13C, the outer product S×G is in forward and backward directions that are perpendicular to the coronal plane SF2. Therefore, when considering an inner product of an arbitrary vector K and the outer product S×G, the inner product takes positive and negative values depending on the direction of the outer product S×G. In other words, when an angle formed between both vectors is less than 90°, the inner product takes a positive value, and when the angle is greater than 90°, the inner product takes a negative value. Therefore, by selecting a vector that is not in a perpendicular relationship with the outer product S×G as the arbitrary vector K, a sign of the inner product of the arbitrary vector K and the outer product S×G can be used as the coefficients +1 and −1 described earlier.

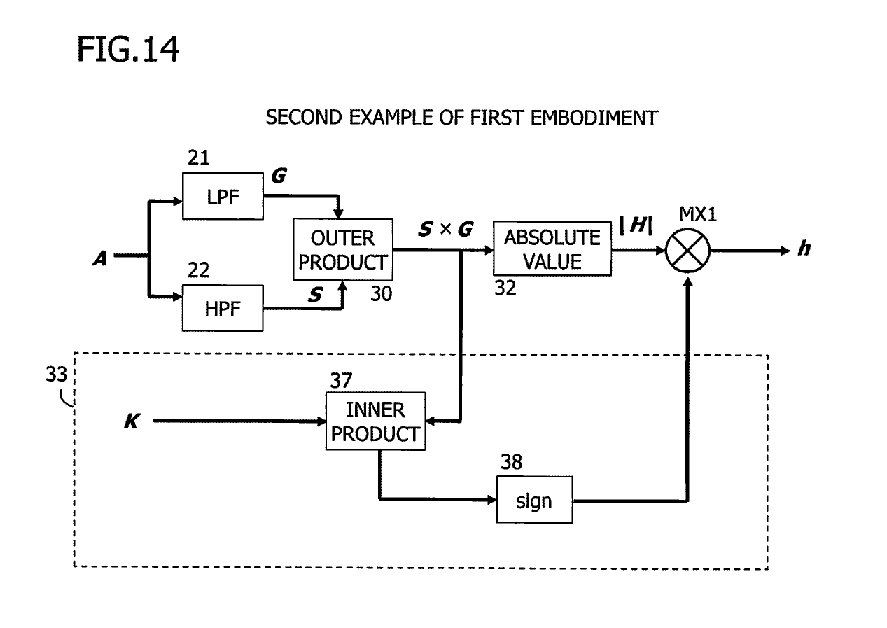

FIG. 14 is a sequence diagram illustrating a computing process for obtaining acceleration in one and the other horizontal directions in the acceleration sensor output processing program according to the second example of the first embodiment. In FIG. 14, the LPF 21, the HPF 22, the outer product computation processing unit 30, the absolute value computation processing unit 32, and the multiplier MX1 are the same as those in FIG. 11. In addition, the computation processing unit 33 that generates the coefficients +1 and −1 include an inner product computation processing unit 37 that computes an inner product of the outer product S×G and the arbitrary vector K and a sign determination processing unit 38. Outputs +1 and −1 of the sign determination processing unit 38 are input to the multiplier MX1.

According to the second example, since a magnitude |v| of acceleration of a vertical movement is not utilized, a magnitude of acceleration in one and the other horizontal directions can be obtained even if the vertical movement and a horizontal movement are out of phase.

The computing process of the second example illustrated in FIG. 14 determines an odd-numbered walking period and an even-numbered walking period of walking based on a sign of an inner product of an outer product (S×G) and an arbitrary vector (K) in the determination step (the computation processing unit 33) in the computing process illustrated in FIG. 8 described earlier.

The computing process described above is executed when the sub microprocessor 14 executes the sensor output processing program 15.

### Third Example of First Embodiment

In the example illustrated in FIG. 14, the arbitrary vector K is necessary to be a vector that is not in a perpendicular relationship with the outer product S×G. However, when the vector K coincidentally exists in a coronal plane, the inner product of the vector K and the outer product S×G equals 0 and a sign cannot be determined. In consideration thereof, as an improvement of FIG. 14, an example will be described in which the coefficients +1 and −1 are generated using three mutually different vectors K₁, K₂, and K₃ which are not on a same plane.

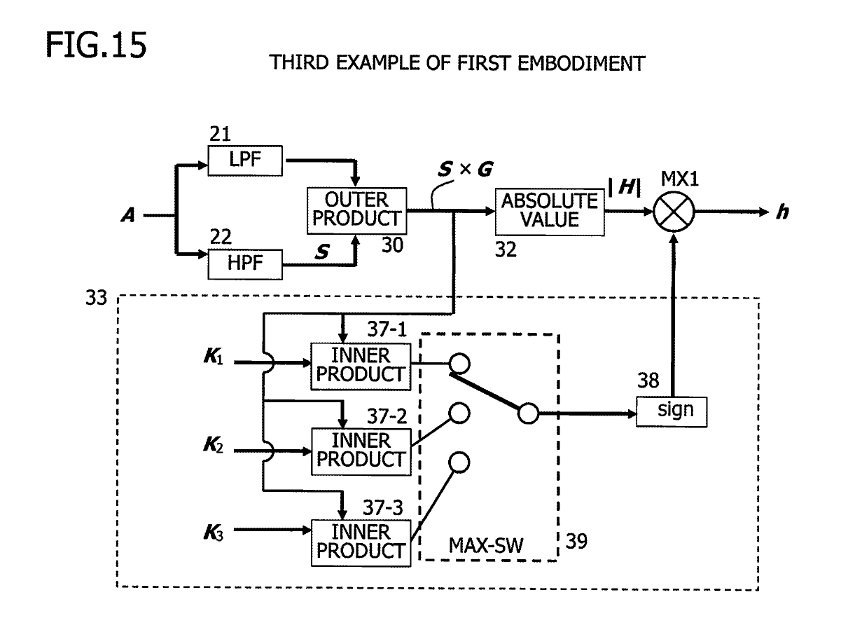

FIG. 15 is a sequence diagram illustrating a computing process for obtaining acceleration in one and the other horizontal directions in the acceleration sensor output processing program according to the third example of the first embodiment. Unlike FIG. 14, in this example, the coefficient computation processing unit 33 that generates the coefficients +1 and −1 corresponding to an odd-numbered walking period and an even-numbered walking period of walking includes inner product computation processing units 37-1, 37-2, and 37-3 that compute inner products of three mutually different vectors K₁, K₂, and K₃ which are not on a same plane and the outer product S×G, as well as a maximum value switch 39 that selects a maximum inner product among the computed inner products. Other components are the same as those illustrated in FIG. 14.

According to this computing process, even if two among the three mutually different vectors K₁, K₂, and K₃ coincidentally exist in a coronal plane SF2, the remaining vector is inevitably a vector having a certain angle with respect to the coronal plane. Therefore, the vector K such as that illustrated in FIG. 13C can be used.

The computing process of the third example illustrated in FIG. 15 determines an odd-numbered walking period and an even-numbered walking period of walking based on a sign of an inner product of a maximum absolute value among inner products of the outer product (S×G) and at least three vectors (K1 to K3) that are not in a same plane in the determination step (the computation processing unit 33) in the computing process illustrated in FIG. 8 described earlier.

The computing process described above is executed when the sub microprocessor 14 executes the sensor output processing program 15.

### Fourth Example of First Embodiment

The second and third examples of the first embodiment described above are premised on the fact that the swing acceleration S does not have a component in the front-back direction and that the swing acceleration S exists in the coronal plane SF2. However, according to data regarding actual walking, acceleration is also generated, albeit in a small magnitude, in the front-back direction due to noise, shock applied when grounding a swinging leg, and the like. Therefore, there may be case where the outer product S×G does not change by a full 180°. In such cases, even if the three mutually different vectors K₁, K₂, and K₃ described above are utilized, depending on the three vectors, it may be unclear as to which of the vectors produces a determination, which represents a different inner product result among the respective vectors, is to be used.

In consideration thereof, in the fourth example of the first embodiment, the computation processing unit 33 that generates coefficients +1 and −1 corresponding to an odd-numbered walking period and an even-numbered walking period of walking generates a forward direction vector Kf (or a backward direction vector) for which the direction of the outer product S×G is switched to one of the front-back direction (forward or backward) and obtains the coefficients +1 and −1 based on a sign of an inner product of the forward direction vector Kf and the outer product S×G. In addition, a magnitude |H| of acceleration in the horizontal direction which represents an absolute value of the outer product S×G is multiplied by the coefficient to generate a signed magnitude h which represents one or the other of left and right.

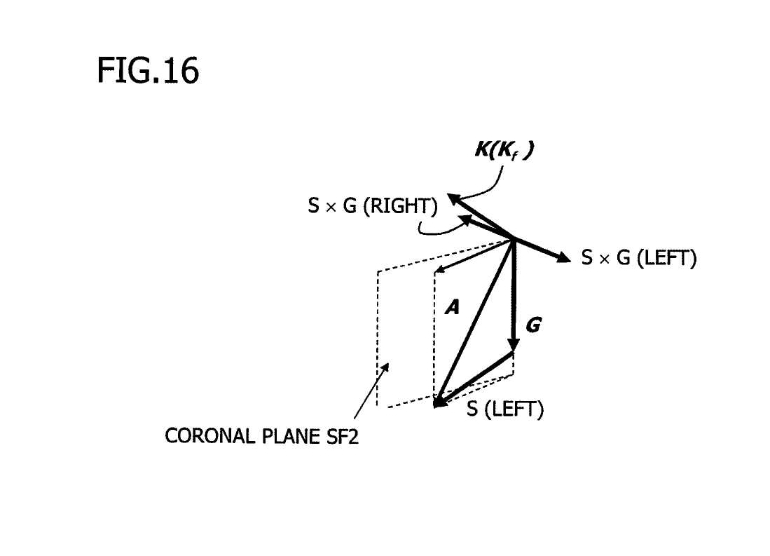

FIG. 16 is a diagram explaining the fourth example of the first embodiment. Unlike FIG. 13C, the swing acceleration S is at a light angle with respect to the coronal plane SF2. This is because the swing acceleration S includes a small front-back direction component.

As described with reference to FIGS. 13A to 13C, the outer product S×G of the swing acceleration S and the gravitational acceleration G points in a forward direction or a backward direction depending on whether the swing acceleration S is in a rightward or leftward direction. Specifically, the outer product S×G points in the backward direction when the swing acceleration S is in the leftward direction and points in the forward direction when the swing acceleration S is in the rightward direction. This tendency remains the same even when the swing acceleration S includes a small front-back direction component. Therefore, for example, by multiplying the outer product S×G by a negative value when the swing component S moves in the leftward direction, a vector Kf that is always oriented forward can be generated. In addition, when obtaining an inner product of the vector Kf that is always oriented forward and the outer product S×G, the inner product is positive if the outer product S×G is oriented forward and the inner product is negative if the outer product S×G is oriented backward. Therefore, by generating the coefficients +1 and −1 using the sign of the inner product result and multiplying the magnitude |H| of acceleration in the horizontal direction which represents an absolute value of the outer product S×G by the coefficients, a magnitude of signed acceleration indicating one or the other of left and right can be obtained.

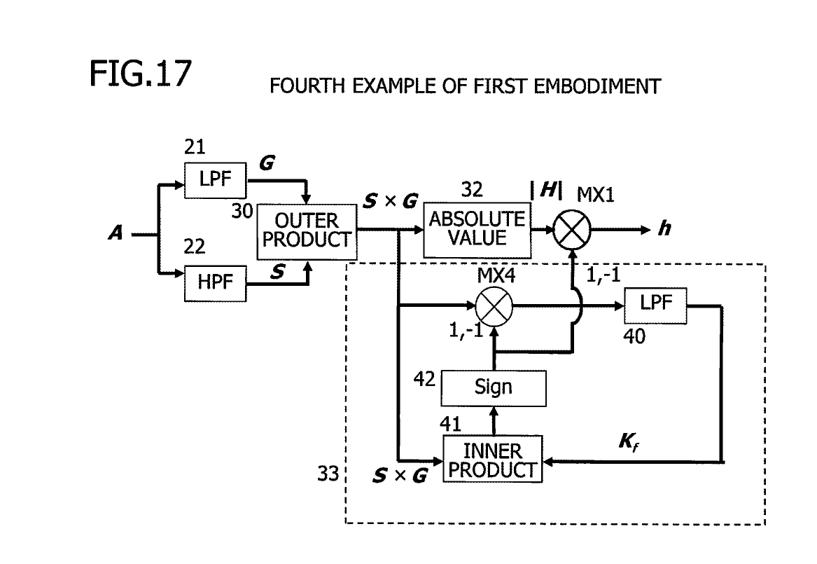

FIG. 17 is a sequence diagram illustrating a computing process for obtaining acceleration in one and the other horizontal directions in the acceleration sensor output processing program according to the fourth example of the first embodiment. In this example, unlike in FIG. 15, the coefficient computation processing unit 33 that generates coefficients +1 and −1 corresponding to an odd-numbered walking period and an even-numbered walking period of walking includes a multiplier MX4, an LPF 40, an inner product computation processing unit 41, and a positive/negative determining unit 42. Other components are the same as those illustrated in FIG. 15.

The coefficient computation processing unit 33 includes a multiplication processing unit MX4 that multiplies the outer product S×G by the coefficients +1 and −1 and the LPF 40 that obtains an average value of output of the multiplication processing unit MX4. Since the outer product S×G is a vector in the front-back direction, by having the multiplication processing unit MX4 multiply the outer product S×G by the coefficients +1 and −1, a vector Kf in one direction of front and back such as the forward direction can be obtained. Subsequently, as the inner product computation processing unit 41 obtains an inner product of the vector Kf in the forward direction and the outer product S×G, the sign of the inner product takes +1 if the outer product S×G is in a forward direction (the swing acceleration S is in a rightward direction) and −1 if the outer product S×G is in a backward direction (the swing acceleration S is in a leftward direction). Therefore, the sign is detected by the sign determining unit 42 and the multiplier MX1 multiplies the magnitude |H| of acceleration in the horizontal direction by +1 or −1 to generate a signed magnitude h of acceleration in one and the other of left and right.

While the coefficient computation processing unit 33 contains an endless loop, it is confirmed that the coefficient computation processing unit 33 operates normally by setting an arbitrary initial value as the vector Kf. In other words, depending on the initial value of the vector Kf, the signed magnitude h of acceleration in one and the other of left and right may take a positive value or a negative value in the rightward direction. Specifically, with respect to h that is computed according to the sequence diagram in FIG. 17, although the rightward direction and the leftward direction cannot be distinguished from each other, a distinction can be made between one and the other of left and right. Therefore, if the obtained magnitude h of one and the other of left and right in the horizontal direction is to be generated by the sub microprocessor, the main microprocessor can execute a gait assessment program to assess a walking state.

The computing process of the fourth example illustrated in FIG. 17 determines an odd-numbered walking period and an even-numbered walking period of walking based on a sign of an inner product of an outer product (S×G) and an arbitrary vector (Kf) and generates the arbitrary vector (Kf) by multiplying the outer product (S×G) by the sign of the inner product and then averaging the result of the multiplication in the determination step (the computation processing unit 33) in the computing process illustrated in FIG. 8 described earlier.

The computing process described above is executed when the sub microprocessor 14 executes the sensor output processing program 15.

[Gait Assessment Program]

Next, before explaining second and third embodiments, an assessment method of a walking state which utilizes the magnitude h of one and the other horizontal directions will be described.

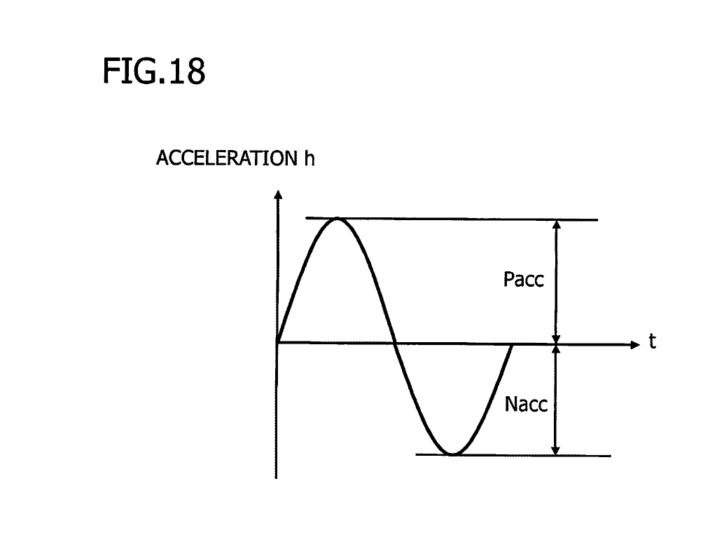

FIG. 18 is a diagram that explains a first example of a gait assessment program. As described earlier, the sub microprocessor 14 generates a magnitude h of acceleration in one and the other of left and right. The magnitude h of acceleration is data having positive and negative magnitudes as time passes as illustrated in FIG. 18.

In consideration thereof, gait assessment is performed based on a ratio between positive maximum acceleration Pacc and negative maximum acceleration Nacc of the magnitude h of acceleration in one and the other in the horizontal direction. For example, an assessment is made that a gait is bilaterally symmetrical when the ratio Pacc/Nacc approaches 1 and that a gait is significantly biased in one direction of left and right when the ratio Pacc/Nacc is significantly larger or smaller than 1.

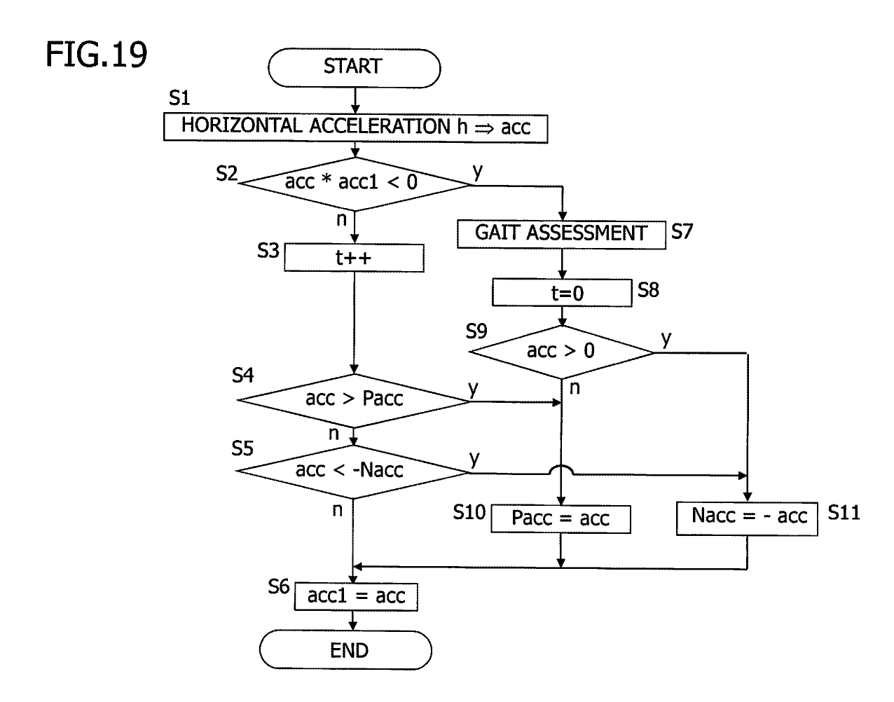

FIG. 19 is a flow chart for obtaining maximum acceleration by a gait assessment program. First, the main microprocessor 11 inputs the magnitude h of acceleration in one and the other horizontal directions which is output from the sub microprocessor 14 (S1), and executes a gait assessment program 12B to obtain maximum acceleration. Steps S1 to S11 in FIG. 19 are repeated each time acceleration h of one and the other horizontal directions is received from the sub microprocessor.

The main processor 11 replaces the received acceleration h with current acceleration acc (S1). Subsequently, a determination is made on whether or not a product acc*acc1 with acceleration acc1 received in a previous cycle is negative (S2). In case of a positive result of step S2 or, in other words, when the acceleration acc has not passed a zero-cross point, time t is set such that t=t+1 (S3), a determination is made on whether or not the current acceleration acc is greater than the stored positive maximum acceleration Pacc (S4), and a determination is made on whether or not the current acceleration acc is smaller than the stored negative maximum acceleration −Nacc (S5). Both Pacc and Nacc denote absolute values of maximum acceleration. When both results of the determinations in steps S4 and S5 are NO, the acceleration acc of the current cycle is replaced with the acceleration acc1 of the previous cycle (S6).

On the other hand, when the product acc*acc1 is found to be negative in step S2 or, in other words, when the acceleration acc has passed a zero-cross point, gait assessment is performed using maximum acceleration Pacc or Nacc obtained in a previous cycle (S7). Subsequently, the time t is reset (S8), when the current acceleration acc is positive (yes in S9), the acceleration acc is replaced with the positive maximum acceleration Pacc (S10), and when the current acceleration acc is negative (no in S9), the acceleration acc is replaced with the negative maximum acceleration Nacc (S11). Moreover, since h is signed, the absolute value Nacc of the negative maximum acceleration is replaced with −acc that is obtaining by multiplying current negative acceleration acc by −1.

Step S10 or S11 described above is executed even when a determination result of yes is obtained in either step S4 or S5 described earlier.

In this manner, a positive maximum value Pacc and a negative maximum value Nacc of the acceleration h are detected for a prescribed period of time.

Moreover, as will be described later, since the time t of an adjacent zero-cross point of the acceleration h is measured in the gait assessment S7, when the time t significantly differs from a period of time that is consumed to switch between left and right legs in an ordinary walking motion, in the gait assessment S7 it is assumed that, for example, the walker is aboard a vehicle or a vessel such as a ship and the maximum acceleration Pacc or Nacc during this period of time is not used.

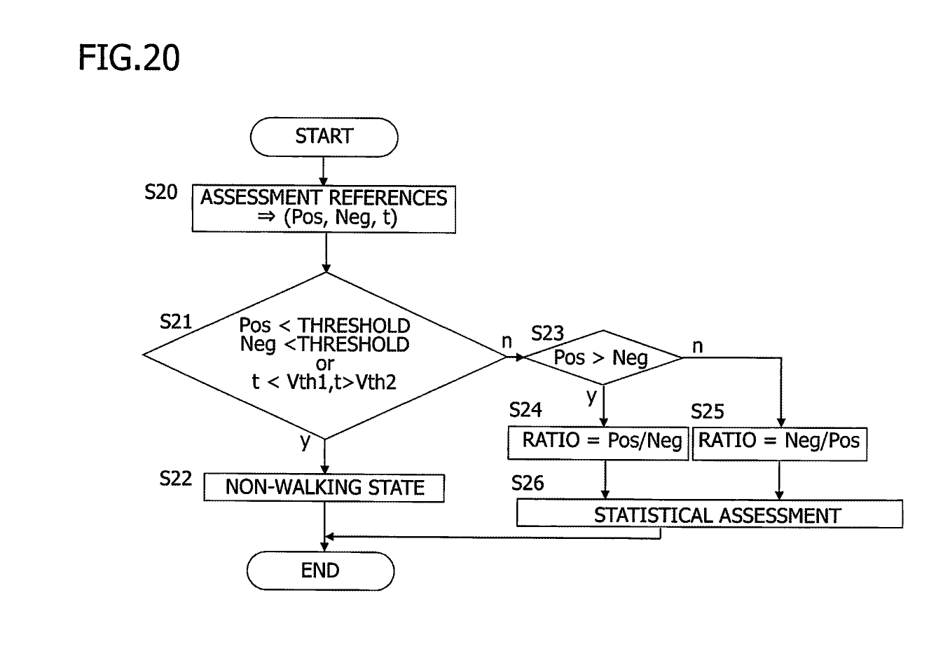

FIG. 20 is a flow chart of a gait assessment according to a gait assessment program that utilizes an acceleration ratio. In the flow chart illustrated in FIG. 20, the maximum accelerations Pacc and Nacc obtained in FIG. 19 are replaced with Pos and Neg. First, the main processor 11 that executes the gait assessment program inputs a positive value Pos, a negative value Neg, and time t as reference values of assessment. The positive value Pos and the negative value Neg are, for example, the maximum accelerations Pacc and Nacc obtained in FIG. 19.

Next, when one of the positive value Pos and the negative value Neg is negative or when the time t is not between two thresholds Vth1 and Vth2 (t<Vth1 and t>Vth2, where Vth1<Vth2) (yes in S21), it is assumed that values do not represent an appropriate walking state and the main processor 11 makes a determination of a non-walking state (S22).

In addition, when the positive value Pos and the negative value Neg are both positive and the time t is such that Vth1<t<Vth2 (no in S21), the positive value Pos and the negative value Neg are compared with each other (S23) and either Pos/Neg or Neg/Pos is obtained as a ratio (S24 and S25). This is performed to facilitate the gait assessment by ensuring that the ratio is equal to or greater than 1.

Subsequently, in step S26, the main processor 11 assesses a walking state based on a ratio of maximum accelerations in the horizontal direction according to a method described later.

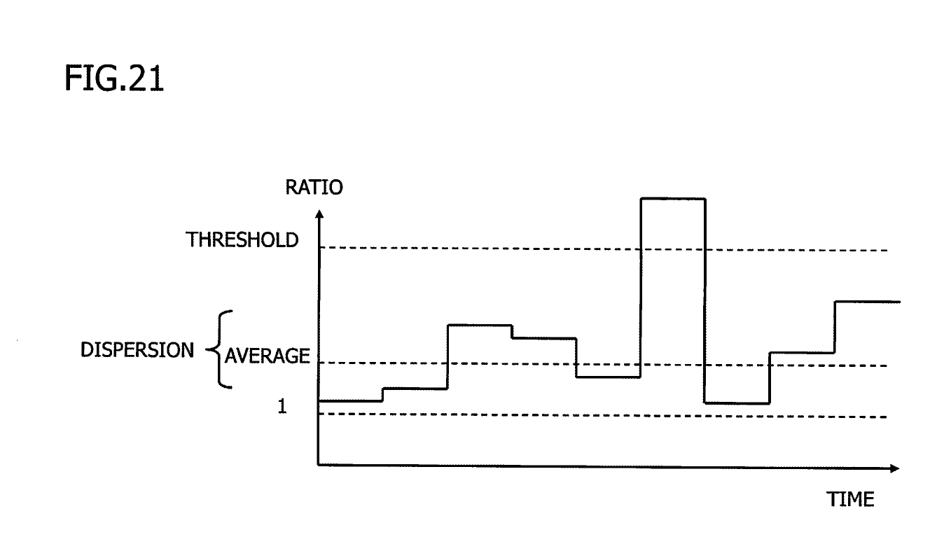

FIG. 21 is a diagram illustrating an example of a transition over time of the ratio obtained in FIG. 20. As illustrated, the ratio takes a value that is equal to or greater than a minimum value of 1 and changes with the lapse of time during walking. In consideration thereof, based on a transition over time of the ratio with respect to walking for a certain period of time (for example, one day), the main processor 11 extracts an average value of the ratio, a dispersion tendency of the ratio, a frequency at which a threshold is exceeded, and the like to perform a gait assessment. For example, when the average value of the ratio is close to 1, an assessment can be made that walking is being performed with favorable left-right balance and, conversely, when the average value of the ratio is greater than 1, an assessment can be made that walking is being performed with unfavorable left-right balance. In addition, an assessment can be made that walking is being performed in a stable manner if dispersion of the ratio is small while an assessment can be made that walking is being performed in an unstable manner if dispersion of the ratio is large. Furthermore, an assessment can be made that a frequency of losing left-right balance is high if the frequency of the threshold being exceeded is high.

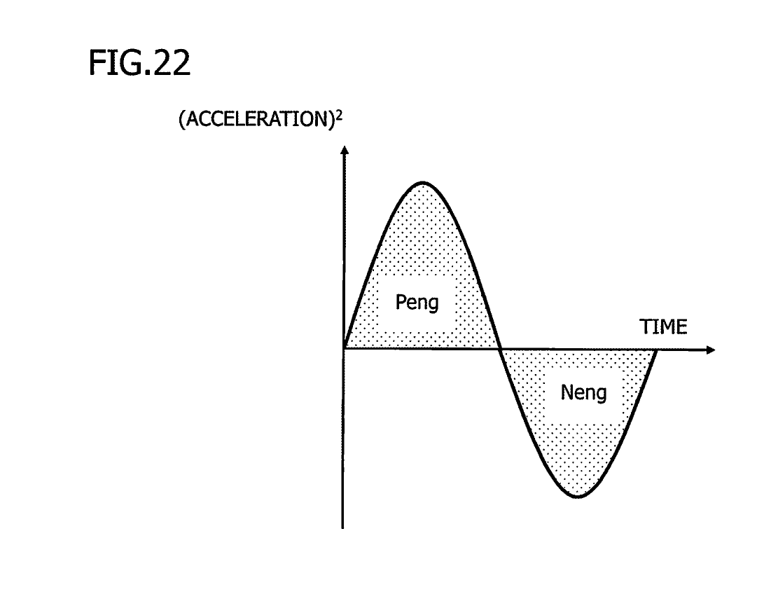

FIG. 22 is a diagram that explains a second example of a gait assessment program. In the second example, a ratio of areas of acceleration h is assessed. The acceleration h is a value obtained by adding a sign of the horizontal direction to the absolute value |H|. In this case, the absolute value is obtained by computing a square root of a sum of squares of elements of a vector. When the computation of a square root is not performed, the acceleration h takes a value of a square of acceleration. FIG. 22 illustrates a change of the square of acceleration. An area of the square of acceleration corresponds to an energy value.

In consideration thereof, in the second example, by accumulating squares of acceleration, the main processor 11 extracts an area Peng of a square of positive acceleration and an area Neng of a square of negative acceleration, obtains a ratio thereof according to the method illustrated in FIG. 20, and performs gait assessment using the ratio.

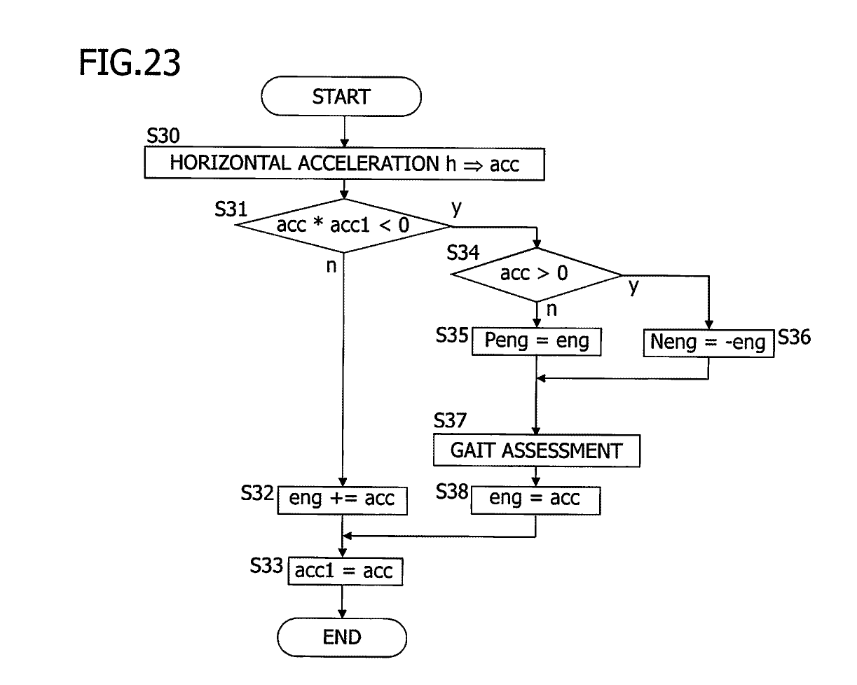

FIG. 23 is a flow chart for extracting areas Peng and Neng of squares of acceleration according to the second example. The main microprocessor 11 executes a program represented by the flow chart illustrated in FIG. 23 in the gait assessment program to extract the areas Peng and Neng of squares of acceleration.

First, the main microprocessor 11 inputs a value h of a square of acceleration as a square acc of current acceleration (S30). Subsequently, the main microprocessor 11 determines whether or not a sign of a product of the square acc of the current acceleration and a value acc1 of a previous cycle is negative (S31). When a result of the determination is yes, since a result of yes means that a zero-cross point has been passed, if the square acc of the current acceleration is negative (no in S34), an accumulated value eng of squares of acceleration that has been accumulated at this point is replaced with the positive area Peng (S35), and if the square acc of the current acceleration is positive (yes in S34), the accumulated value eng of squares of acceleration that has been accumulated at this point is replaced with the negative area Neng (S36). Subsequently, gait assessment is executed based on these values (S37). In addition, as an initial value, the square acc of the current acceleration is substituted for the accumulated value eng (S38).

On the other hand, when the result of the determination in step S31 is no, since a result of no means that a zero-cross point has not been passed, the square acc of the current acceleration is simply added to the accumulated value eng (S32). In addition, the square acc of the current acceleration is substituted for the square acc1 of the previous cycle (S33).

By executing the process illustrated in the flow chart described above, the main microprocessor 11 can extract an area of a square of acceleration illustrated in FIG. 22 for both a positive side Peng and a negative side Neng.

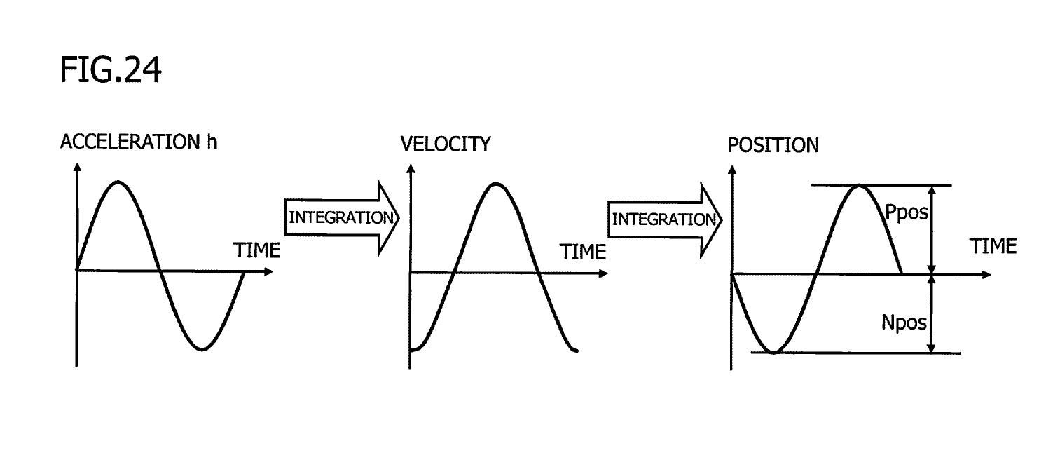

FIG. 24 is a diagram that explains a third example of a gait assessment program. In the third example, the main microprocessor 11 twice integrates acceleration h that is input from the sub microprocessor 14 to extract positions of a walker in one and the other horizontal directions. By assessing a ratio of positions Ppos and Npos in the horizontal direction as illustrated in FIG. 21, a walking state of the walker can be assessed. Specifically, when the ratio is close to 1, an assessment can be made that walking is being performed with favorable left-right balance and when the ratio is greater than 1, an assessment can be made that walking is being performed with unfavorable left-right balance. An average value of the ratio of left and right positions, a dispersion tendency of the ratio, a frequency at which a threshold is exceeded, and the like can be utilized in a similar manner to perform the gait assessment.

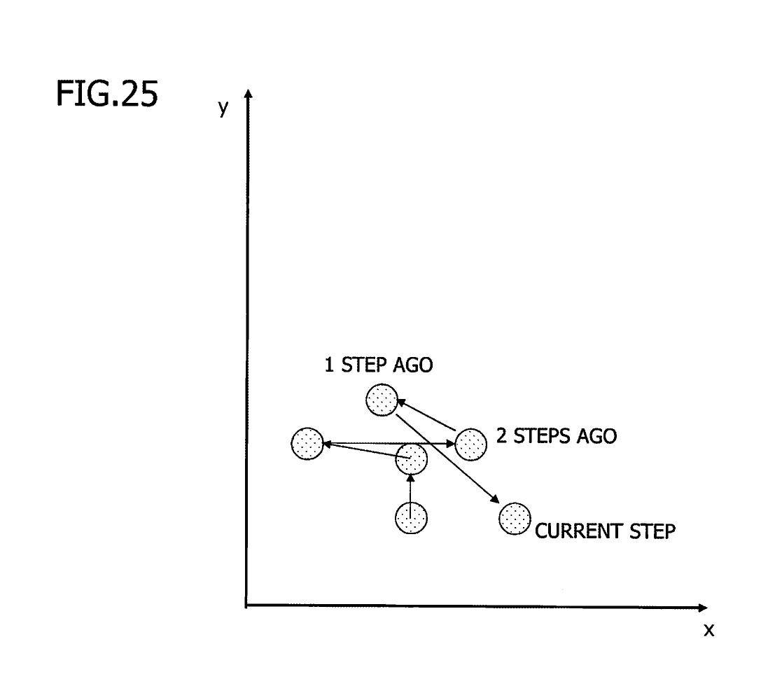

FIG. 25 is a diagram that explains a fourth example of a gait assessment program. In the examples described above, walking is assessed based on acceleration h of one and the other of the horizontal direction. In the fourth example illustrated in FIG. 25, walking is assessed based on two pieces of information, namely, a vertical swing and a lateral swing.

In FIG. 25, combinations of a magnitude |v| of a vertical swing and respective ratios of a maximum value of acceleration h in the horizontal direction, an area of a square of acceleration, a maximum value of positions, and the like are assigned to an x axis and a y axis. For example, a combination of a ratio of maximum value of acceleration and a ratio of areas of squares of acceleration are assigned to the x axis and the y axis. In addition, a coordinate of the combination for each step is plotted in the coordinates. By assessing the plotted points, walking can be assessed.

### Second Embodiment

The first embodiment is premised on a mobile terminal that is mounted to the trunk of a walker hardly being subjected to swinging in the front-back direction in a normal walking state. Based on this premise, the sub microprocessor 14 executing the acceleration sensor output processing program outputs a magnitude of the outer product S×G of swing acceleration S and gravitational acceleration G as a magnitude h of acceleration in one and the other horizontal directions.

However, in exceptional cases, when the walker has placed the mobile terminal in a breast pocket of clothing, a front pocket of slacks, and the like, the mobile terminal swings forward and backward like a pendulum and acceleration in the front-back direction is observed.

In consideration thereof, in the second embodiment, the sub microprocessor 14 that executes an acceleration sensor output processing program calculates a magnitude of acceleration in one and the other horizontal directions. Furthermore, in addition to the magnitude of acceleration in one and the other horizontal directions, the sub microprocessor 14 may calculate a magnitude of acceleration in one and the other front-back directions. However, a distinction between a leftward direction and a rightward direction and a distinction between a forward direction and a backward direction cannot be made.

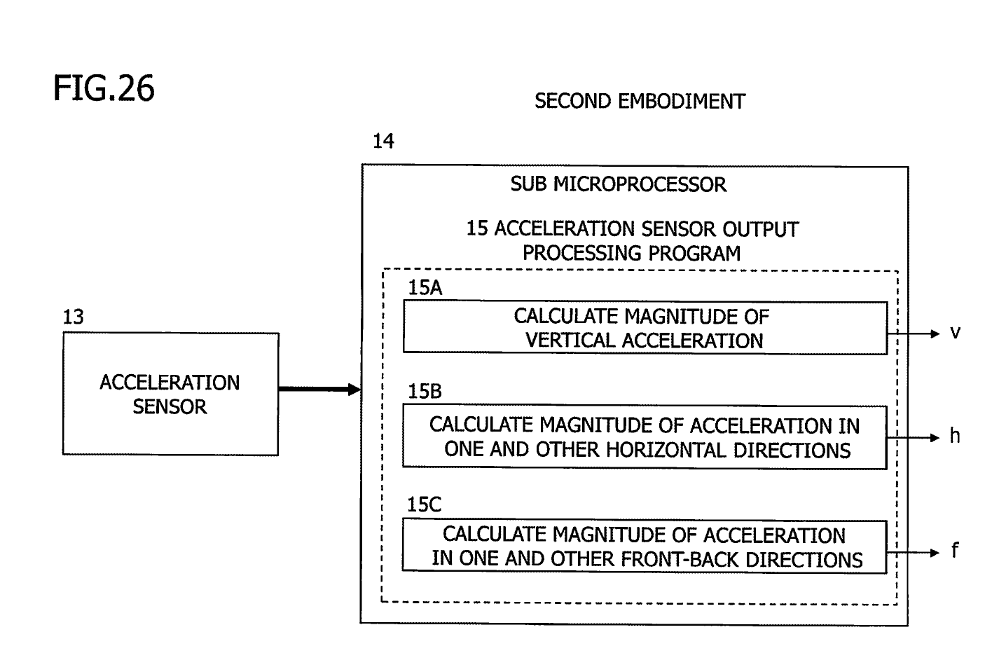

FIG. 26 is a diagram illustrating a function of an acceleration sensor output processing program according to the second embodiment. The acceleration sensor output processing program 15 includes a function 15A for calculating a magnitude v of acceleration in the vertical direction, a function 15B for calculating a magnitude h of acceleration in one and the other horizontal directions, and a function 15C for calculating a magnitude f of acceleration in one and the other front-back directions.

An output of an acceleration sensor in a walking state is a composite of acceleration with respect to movement in the horizontal direction, acceleration with respect to movement in the vertical direction, and, albeit small, acceleration of movement in the front-back direction. In addition, according to the walking state illustrated in FIG. 2, a period of horizontal movement is approximately twice as long as a period of vertical movement. In this manner, since frequencies of horizontal movement and vertical movement differ from each other, by utilizing different band pass filters having passing frequency band characteristics that respectively correspond to the frequencies, acceleration of the horizontal movement and acceleration of the vertical movement can be separated from the sensor output A. The vertical movement may be assumed to be equal to a walking period while the horizontal period may be assumed to be twice as long. According to a gait of an average person, the frequency of the vertical movement can be set to 2 Hz and the frequency of the horizontal movement can be set to 1 Hz. Alternatively, the frequencies may be set to optimal frequencies according to a height, walking record, and the like of the person who is an object of gait assessment.

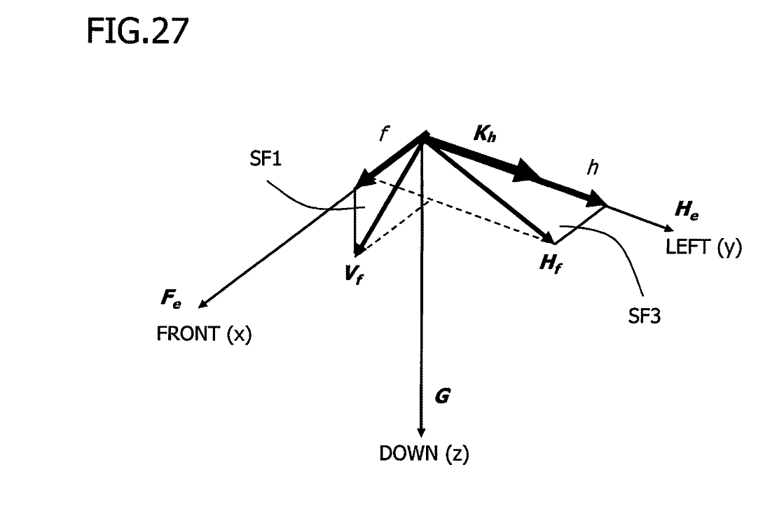

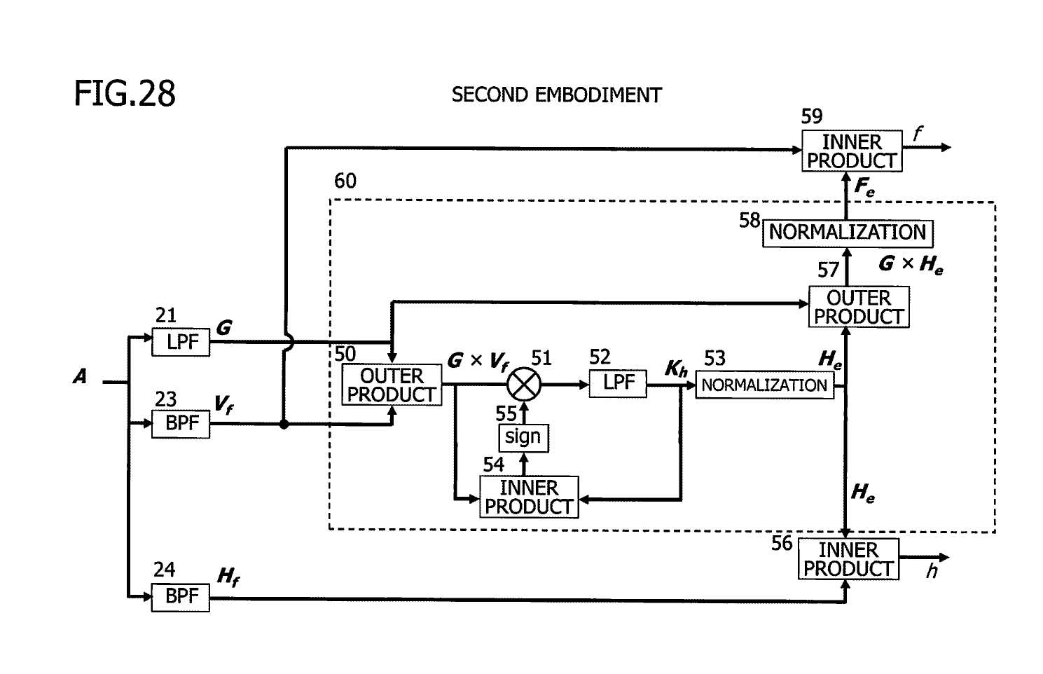

FIG. 27 is a diagram illustrating a relationship between acceleration Hf in the horizontal direction and acceleration Vf in the vertical direction which have been separated by band pass filters according to the principles described above. FIG. 28 is a sequence diagram illustrating a computing process for obtaining a magnitude of acceleration in one and the other horizontal directions and a magnitude of acceleration in one and the other front-back directions in the acceleration sensor output processing program according to the second embodiment.

The sequence diagram includes: a low pass filter 21 that extracts gravity direction acceleration G; a first band pass filter (BPF1) 23 that extracts front-back direction acceleration Vf, and a second band pass filter (BPF2) 24 that extracts acceleration Hf in the horizontal direction from a sensor output A; a unit vector calculating unit 60 that generates a unit vector He in one of the horizontal direction and a unit vector Fe in one of the front-back direction; and inner product processing units 56 and 59.

In FIG. 27, both the acceleration Hf in the horizontal direction and the acceleration Vf in the vertical direction include, albeit slightly, acceleration in the front-back direction.

However, due to the operations of the two band pass filters (BPF1) 23 and (BPF2) 24 illustrated in FIG. 28, the acceleration Hf in the horizontal direction includes a component in the front-back direction but does not include a component in the vertical direction. Therefore, the acceleration Hf in the horizontal direction is in a plane (the transverse plane SF3) constituted by the x axis and the y axis. In a similar manner the acceleration Vf in the vertical direction includes a component in the front-back direction but does not include a component in the horizontal direction. In other words, the acceleration Vf in the vertical direction is in a plane (the sagittal plane SF1) constituted by the x axis and the z axis.

In addition, the acceleration component G in the gravity direction can be extracted from an output of the acceleration sensor by a low pass filter (LPF) 21 having a significantly lower cut-off frequency. Therefore, as illustrated in FIG. 27, an outer product G×Vf of the gravity direction component G and the acceleration Vf in the vertical direction is a vector on the y axis that is a line of intersection of the transverse plane SF3 and the coronal plane SF2. In FIG. 27, since the acceleration Vf in the vertical direction has a forward direction component, the outer product G×Vf is oriented in the rightward direction. When the acceleration Vf in the vertical direction has a backward direction component, the outer product G×Vf is to be oriented in the leftward direction.

In consideration thereof, in a similar manner to the example illustrated in FIG. 17, the unit vector calculating unit 60 illustrated in FIG. 28 uses the outer product processing unit 50 to obtain the outer product G×Vf, multiplies (the multiplier 51) the outer product G×Vf by the coefficients +1 and −1 at timings of changes in the front-back direction, and generates a vector Kh in one of the horizontal directions which is illustrated in FIG. 27. This vector Kh is a horizontal direction reference vector having one of the horizontal directions and a magnitude of a constant average value. While the vector Kh is pointed to the left in FIG. 27, this simply corresponds to a case where the direction of the outer product G×Vf is reversed by the multiplier 51. Subsequently, based on whether an inner product (the inner product processing unit 54) of the vector Kh and the outer product G×Vf is positive or negative, a determination (the sign determination processing unit 55) is made on whether the outer product G×Vf is oriented in one or the other of the leftward direction and the rightward direction to obtain the coefficients described earlier. The low pass filter 52 illustrated in FIG. 28 has a function for obtaining an average value.

In addition, while the vector Kh is oriented in one of the horizontal directions, since its magnitude depends on a magnitude of movement Vf in the front-back direction, the magnitude of the vector Kh is normalized to generate a unit vector He by the normalization processing unit 53.

Furthermore, when an outer product G×He of the gravity direction acceleration vector G and the vector He is obtained by the outer product processing unit 57, the outer product G×He is a vector on the x axis that is a line of intersection of the sagittal plane SF1 and the transverse plane SF3 as shown in FIG. 27. The direction of the vector G×He is oriented in either the forward or backward direction. In other words, the vector G×He is a front-back direction reference vector having one of the front-back directions and a magnitude of a constant average value. Subsequently, the magnitude of the outer product G×He that is the reference vector is normalized (normalization processing unit 58) to obtain a unit vector Fe in the forward direction.

By generating an inner product (inner product processing unit 56) of the unit vector He in the horizontal direction that is calculated by the unit vector calculating unit 60 as described above and the acceleration vector Hf in the horizontal direction, a magnitude h of acceleration which enables a distinction to be made between one and the other horizontal directions is generated. As illustrated in FIG. 27, h is a pure magnitude of acceleration in the horizontal direction which corresponds to a magnitude on the y axis of the acceleration Hf in the horizontal direction and which does not include a component in the front-back direction.

In a similar manner, by generating an inner product (inner product processing unit 59) of the unit vector Fe in the front-back direction and the acceleration vector Vf in the vertical direction, a magnitude f of acceleration which enables a distinction to be made between one and the other front-back directions is generated. Similarly, f is a pure magnitude of acceleration in the front-back direction which corresponds to a magnitude on the x axis of the acceleration Vf in the front-back direction and which does not include a component in the vertical direction.

While the magnitude h of one and the other horizontal directions is oriented in the leftward direction in FIG. 27, the magnitude h may possibly be oriented in the rightward direction. In addition, while the magnitude f of one and the other front-back directions is oriented in the forward direction, the magnitude f may possibly be oriented in the backward direction. In this manner, the magnitudes h and f which are obtained as inner products using the unit vectors He and Fe take positive and negative values and enable one and the other in the horizontal direction or the front-back direction to be distinguished from each other. However, it is unclear as to which of the horizontal directions or which of the front-back directions the magnitudes h and f are oriented in. This is because, with respect to a loop involving the outer product G×Vf and the vector Kh that corrects the outer product G×Vf in a given direction in the unit vector calculating unit 60, directions of the unit vectors He and Fe change depending on which direction the initial value Kh is set to. Furthermore, the direction of the unit vectors He and Fe also differ depending on an order of outer product computations by the outer product processing units 50 and 57.

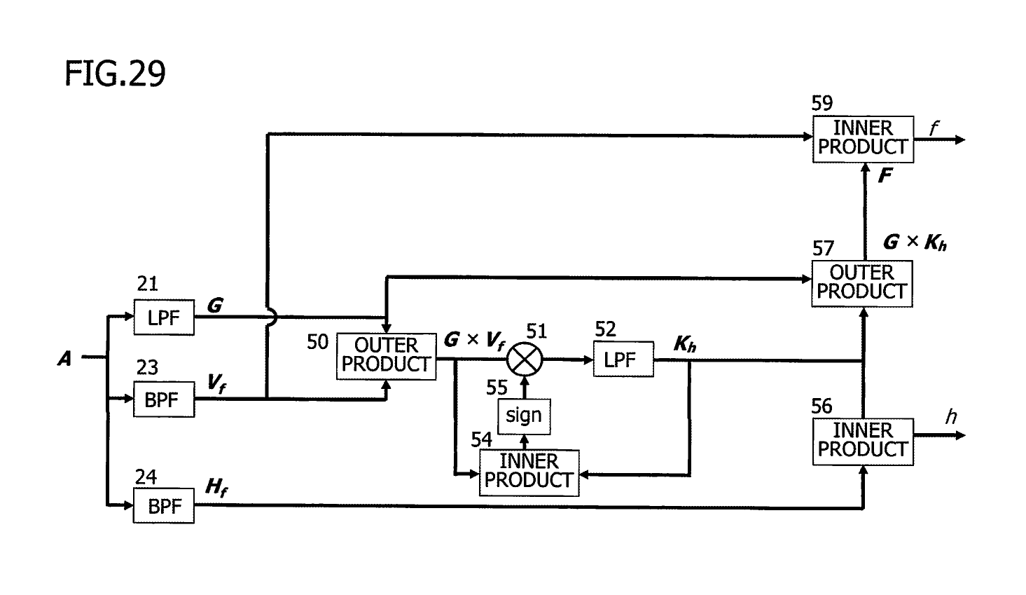

FIG. 29 is a sequence diagram illustrating a computing process for obtaining acceleration in one and the other horizontal directions and acceleration in one and the other front-back directions in the acceleration sensor output processing program according to the second embodiment. FIG. 29 represents a modification of FIG. 28. The configuration illustrated in FIG. 29 differs from that illustrated in FIG. 28 in that the normalization processing units 53 and 58 are not provided. Otherwise, the configuration is similar to that illustrated in FIG. 28. Accordingly, a vector Kh on the y axis is utilized instead of the unit vector He and a vector F on the x axis is utilized instead of the unit vector Fe.

A normalization process requires (includes) a computing process of obtaining square roots of respectively squared value of x, y, and z components of a vector and dividing the square roots into unit magnitudes. In consideration thereof, the example illustrated in FIG. 29 is not provided with a normalization processing unit. In FIG. 29, since the vector Kh is obtained by being passed through the LPF 52 and the vector F is also obtained by being passed through the LPF 58A between the units 57 and 59, by sufficiently increasing time constants of the LPFs, magnitudes of the vectors are averaged and takes constant values. Specifically, since the vectors Kh and F are constant multiples of the unit vectors He and Fe, a magnitude h of acceleration in the horizontal direction and a magnitude f of acceleration in the front-back direction which are obtained by inner products are constant multiples of acceleration.

However, since the gait assessment described earlier does not utilize absolute magnitudes of the magnitude h of acceleration in the horizontal direction and the magnitude f of acceleration in the front-back direction but utilizes a ratio that represents a comparison between the magnitude h and the magnitude f, the fact that the magnitudes are multiplied by constants does not pose any problems.

In FIGS. 28 and 29 which illustrate the second embodiment, only the magnitude h of acceleration in the horizontal direction may be generated. Conversely, only the magnitude f of acceleration in the front-back direction may be generated in FIGS. 28 and 29. Furthermore, both magnitudes h and f may be generated.

The computing process illustrated in FIGS. 28 and 29 is performed by the following steps.

Specifically, an acceleration sensor output process includes the steps of: calculating an outer product of a gravity component vector G extracted using a low pass filter 21 and a front-back swing component vector Vf extracted using a first band pass filter 23 from a sensor value A to generate a first outer product G×Vf;
generating horizontal reference vector Kh or He having one of the horizontal directions and constant magnitudes by multiplying the first outer product G×Vf by a prescribed alternately-changing sign and averaging thereof;
generating a prescribed sign based on a sign of a first inner product of the first outer product G×Vf and the horizontal reference vectors Kh and He; and
calculating an inner product of a horizontal swing component vector Hf extracted using a second band pass filter 24 whose passing frequency band is lower than that of the first band pass filter 23 from the sensor value A, and the horizontal reference vector He it Kh to generate a first magnitude h of acceleration in one and the other horizontal directions.

In addition, the acceleration sensor output process further includes the steps of: calculating an outer product G×He or G×Kh of the gravity component vector G and the horizontal reference vector He or Kh to generate a front-back reference vector Fe or F having one of the front-back directions and a constant magnitude; and calculating an inner product of the front-back swing component vector Vf and the front-back reference vector Fe or F to generate a second magnitude f of acceleration in one and the other front-back directions.

The processing described above is executed when the sub microprocessor 14 executes the sensor output processing program 15.

### Third embodiment

In the second embodiment described above, while the sub microprocessor 14 that executes an acceleration sensor output processing program calculates a magnitude of acceleration in one and the other front-back directions in addition to the magnitude of acceleration in one and the other horizontal directions, a distinction between a leftward direction and a rightward direction and a distinction between a forward direction and a backward direction cannot be made. By comparison, the third embodiment enables a distinction between a leftward direction and a rightward direction and a distinction between a forward direction and a backward direction to be made.

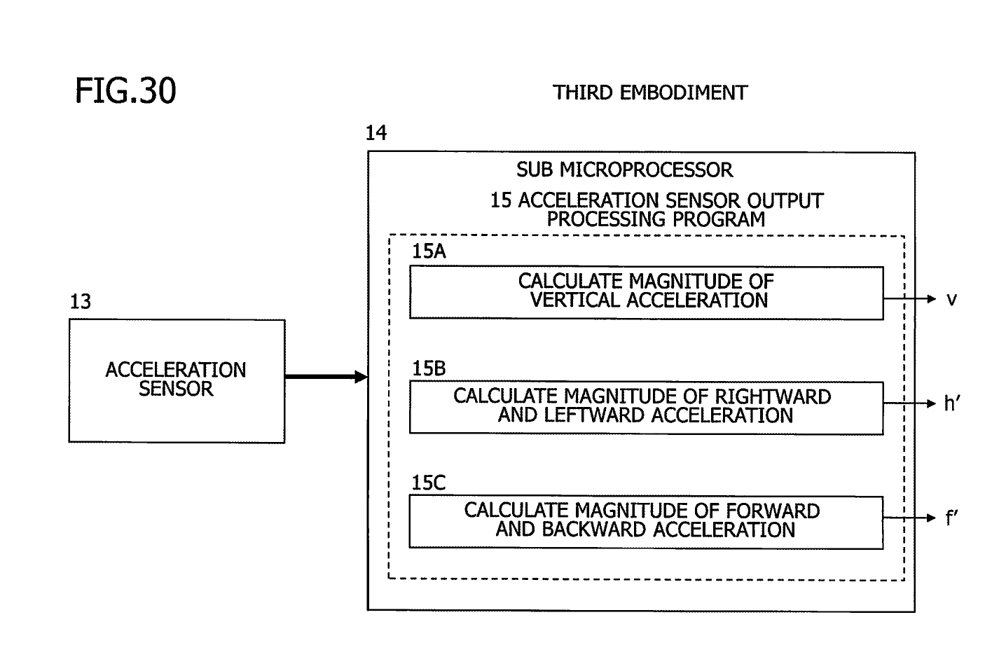

FIG. 30 is a diagram illustrating a function of an acceleration sensor output processing program according to the third embodiment. The acceleration sensor output processing program 15 includes a function 15A for calculating a magnitude v of acceleration in the vertical direction, a function 15B for calculating a magnitude h′ of acceleration in the horizontal direction, and a function 15C for calculating a magnitude f′ of acceleration in the front-back direction. v enables a distinction to be made between upward and downward, h′ enables a distinction to be made between leftward and rightward, and f′ enables a distinction to be made between forward and backward.

The magnitude h of acceleration in one and the other horizontal directions and the magnitude f of acceleration in one and the other front-back directions which are generated in FIGS. 28 and 29 are dependent on initial values of a computing process, an order of outer product computations, and the like and do not enable a distinction to be made between directions. In comparison, in the third embodiment, a phenomenon in which a vertical movement and a front-back movement of a walker becomes a combination of upward and forward or downward and backward is utilized to modify a sign of the magnitude h of acceleration in one and the other horizontal directions and a sign of the magnitude f of acceleration in one and the other front-back directions to correct signs that correspond to the horizontal direction and the front-back direction. Due to the modification, the magnitude h′ of acceleration that enables a distinction to be made between horizontal directions and the magnitude f′ of acceleration that enables a distinction to be made between front-back directions can be correctly generated.

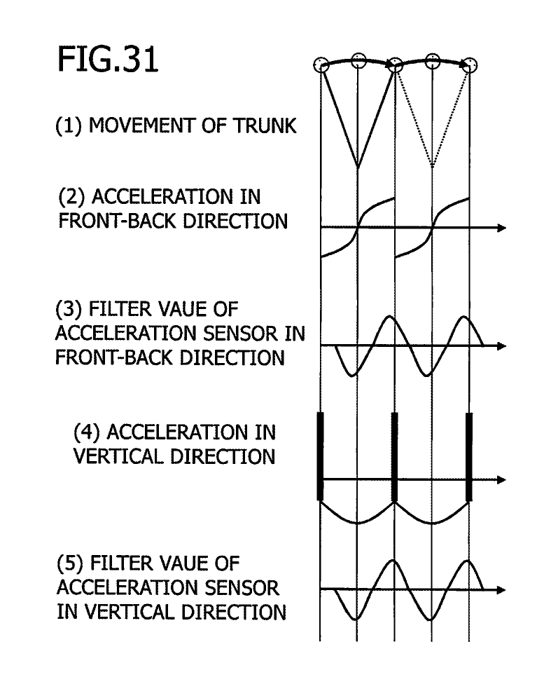

FIG. 31 is a diagram illustrating a relationship between a vertical movement and a front-back movement in walking. (1) of FIG. 31 illustrates a movement of the trunk. As illustrated, since the trunk slightly vibrates in the vertical direction, potential energy changes depending on vertical movement. On the other hand, since mechanical energy retained in the trunk does not change, the changed potential energy is converted to kinetic energy without modification. Since kinetic energy refers to velocity, acceleration in the front-back direction repetitively changes from negative to positive as illustrated in (2) of FIG. 31. (3) of FIG. 31 illustrates a waveform of acceleration in the front-back direction after being smoothed by a sensor filter.

On the other hand, as illustrated in (4) of FIG. 31, acceleration in the vertical direction takes a large positive value when switching supporting legs and subsequently takes a negative value. (5) of FIG. 31 illustrates acceleration in the vertical direction after being smoothed by a sensor filter. Moreover, output of the sensor filter has a prescribed phase delay. Phases in (3) and (5) of FIG. 31 are adjusted to a same phase.

As illustrated in FIG. 31, when the trunk ascends, the velocity of the trunk decreases and forward acceleration takes a negative value, and when the trunk descends, the velocity of the trunk increases and forward acceleration takes a positive value. In addition, with respect to the vertical direction, downward acceleration takes a positive value when the trunk descends and until contact is made with the ground, and downward acceleration takes a negative value when the trunk ascends after contact with the ground.

As described above, a comparison between (3) and (5) of FIG. 31 reveals that periods of acceleration change in the vertical direction and the front-back direction are consistent with each other. It is also revealed that acceleration in the front-back direction is positive when acceleration in the vertical direction is positive, and acceleration in the front-back direction is negative when acceleration in the vertical direction is negative. Furthermore, a vertical orientation can be determined by a comparison with the direction of gravity. Using these characteristics of a walking motion, in the third embodiment, a magnitude of acceleration that enables a distinction to be made between horizontal directions and a magnitude of acceleration that enables a distinction to be made between front-back directions are generated.

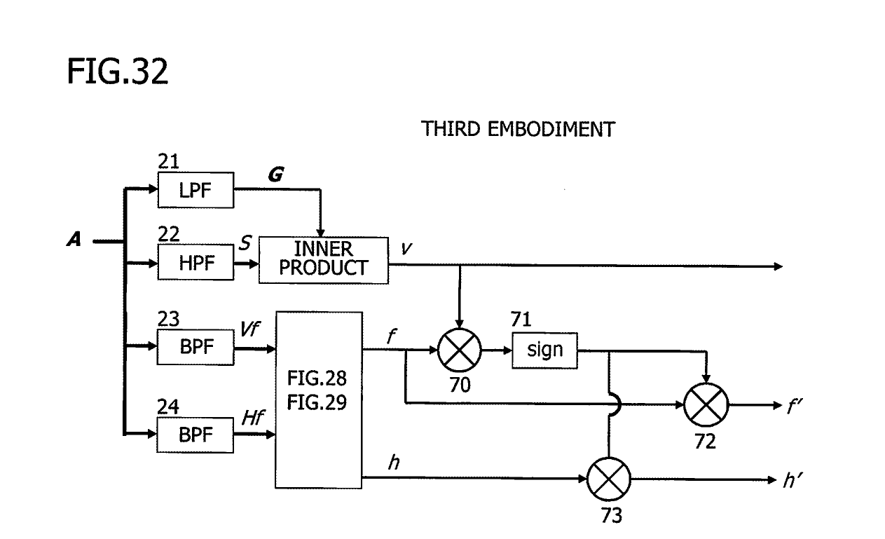

FIG. 32 is a sequence diagram illustrating a computing process for obtaining a magnitude of acceleration in the horizontal direction and a magnitude of acceleration in the front-back direction in the acceleration sensor output processing program according to the third embodiment. In the sequence diagram, using these characteristics of a walking motion, a sign of the magnitude f of acceleration in the front-back direction and a sign of the magnitude h of acceleration in the horizontal direction obtained in FIGS. 28 and 29 are modified to correct signs.

Specifically, in the acceleration sensor output processing program, acceleration v in the vertical direction and acceleration f in the front-back direction are multiplied by a multiplier 70. Since a positive multiplication value signifies that the signs of v and f are in phase (both positive or both negative), it is revealed that the sign of the acceleration f in the front-back direction is correct and that a positive f signifies the forward direction and a negative f signifies the backward direction. Therefore, f′ is generated by a multiplier 72 without inverting the sign of the acceleration f in the front-back direction. On the other hand, since a negative multiplication value of the multiplier 70 signifies that the signs of v and f are in opposite phases (positive and negative or negative and positive), it is revealed that the sign of f is opposite. Therefore, f′ is generated by the multiplier 72 by inverting the sign of the acceleration f in the front-back direction. Accordingly, the modified acceleration f′ in the front-back direction signifies the forward direction when positive and signifies the backward direction when negative.

Furthermore, by modifying the sign of the acceleration h in the horizontal direction with a multiplier 73 using the sign of the multiplication value of the multiplier 70, a modified h′ signifies the leftward direction when positive and the rightward direction when negative and enables a distinction to be made between horizontal directions. This is because, in FIGS. 28 and 29, since the vector Kh is either oriented leftward or rightward depending on an initial value or the like, directions of the unit vectors He and Fe are not fixed and, as a result, a direction signified by the sign of the acceleration f in the front-back direction and a direction signified by the sign of the acceleration h in the horizontal direction are correct in some cases and incorrect in other cases. In other words, depending on the direction of the vector Kh, directions of the unit vectors He and Fe differ and, accordingly, directions signified by signs of the acceleration f in the front-back direction and the acceleration h in the horizontal direction are uncertain. In consideration thereof, it is obvious from FIG. 27 that, when the modified acceleration f′ in the front-back direction signifies the forward direction when positive and signifies the backward direction when negative, the unit vector Fe is in the forward direction, and since the unit vector Fe is in the same direction as the outer product G×He, the unit vector He is in the leftward direction. Therefore, h′ that is modified by the sign of the multiplication value of the multiplier 70 signifies the leftward direction when positive and the rightward direction when negative.

However, when an order of outer product computation differs, the modified h′ signifies the rightward direction when positive and the leftward direction when negative. Therefore, positive and negative directions of f′ and h′ which have been modified depending on a computing process can be uniformly determined.

As described above, in FIG. 32, the multiplier 70 multiplies a vertical magnitude v and a front-back magnitude f, modifies a sign of a magnitude h of acceleration in one and the other horizontal directions and a sign of the magnitude f of acceleration in one and the other front-back directions according to a sign of the multiplication result, to generate h′ and f′. Accordingly, the modified h′ can be distinguished as being in the leftward direction when positive and being in the rightward direction when negative, and the modified f′ can be distinguished as being in the forward direction when positive and being in the backward direction when negative.

The computing process illustrated in FIG. 32 includes the following processing steps. Specifically, in addition to the computing process illustrated in FIGS. 28 and 29, the computing process illustrated in FIG. 32 includes the step of: modifying a sign of a first magnitude h based on a sign of a second inner product v of a swing component vector S extracted by a high pass filter from a sensor value A and the gravity component vector G to generate a first magnitude h′ that enables horizontal directions to be distinguished from each other.

Furthermore, in addition to the computing process illustrated in FIGS. 28 and 29, the computing process illustrated in FIG. 32 includes the step of: modifying a sign of a second magnitude f based on a sign of a second inner product v of the swing component vector S extracted by a high pass filter from the sensor value A and the gravity component vector G to generate a second magnitude f′ that enables front-back directions to be distinguished from each other.

[Processing of LPF, HPF, and BPF]

Computations of the LPF, HPF, and BPF are performed in the embodiments described above. In consideration thereof, computing processes by these filters will now be described.

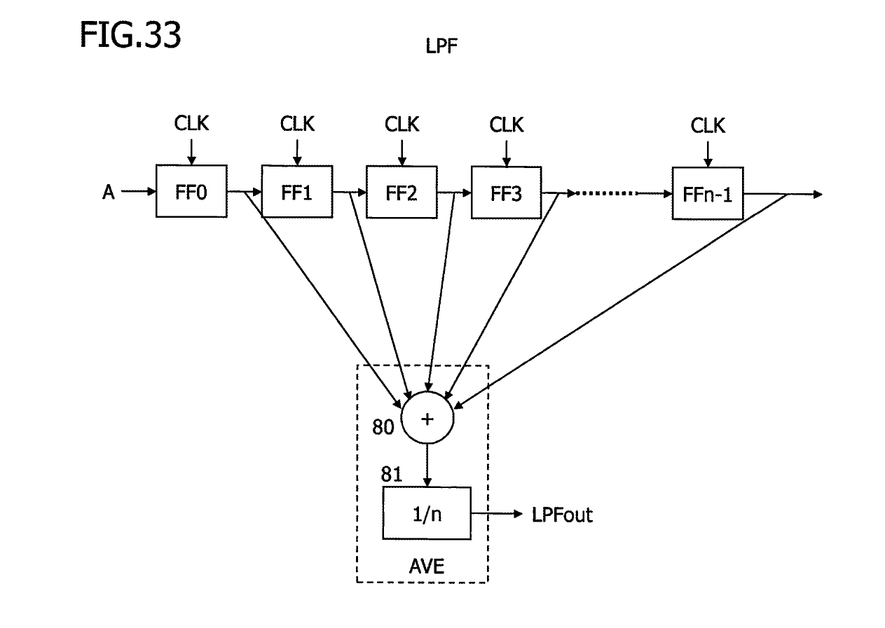

FIG. 33 is a sequence diagram of a computing process by the low pass filter LPF. An acceleration sensor value A is stored in a plurality of flip-flops FF0 to FFn−1 in synchronization with a clock CLK that is synchronized with a sampling clock of the acceleration sensor. In addition, by an averaging process AVE which includes an adder 80 and a divider 81 that performs multiplication by 1/n, an average value of n-number of consecutive sensor values A is obtained. In other words, the low pass filter LPF can be realized by executing an averaging process for obtaining an average of n-number of sensor values A. Therefore, when executing the acceleration sensor output processing program 15, the sub microprocessor 14 executes a computing process of the low pass filter by sequentially storing the sensor values A in a memory, adding up previous n-number of sensor values A, and dividing the sum by n. Accordingly, the sub microprocessor 14 can realize an extraction process by an LPF with the acceleration sensor output processing program 15.

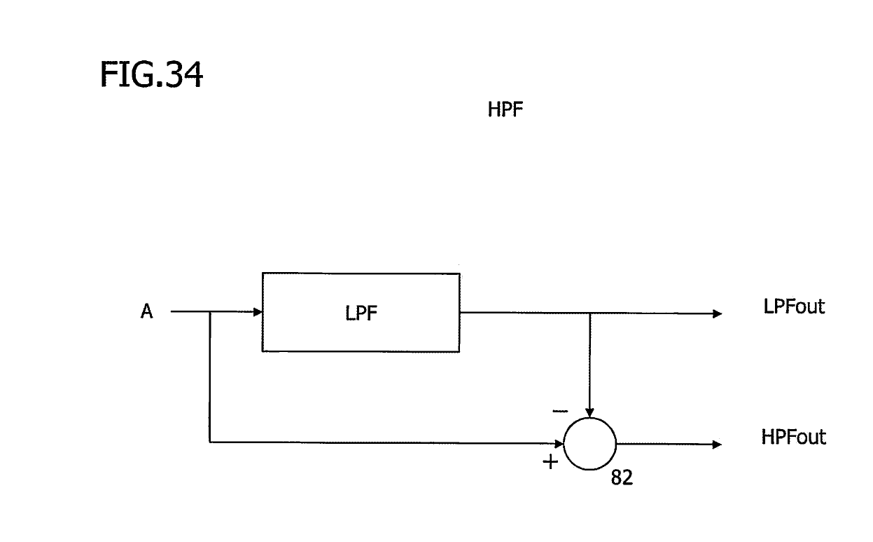

FIG. 34 is a sequence diagram of a computing process by the high pass filter HPF. The computing process by the high pass filter is realized by using a subtracter 82 to subtract an output LPFout of the low pass filter obtained in FIG. 33 from the sensor value A. Therefore, the sub microprocessor 14 may perform a subtracting computation of the low pass filter output LPFout from the sensor value A. An extraction process by the HPF is be realized with the acceleration sensor output processing program 15.

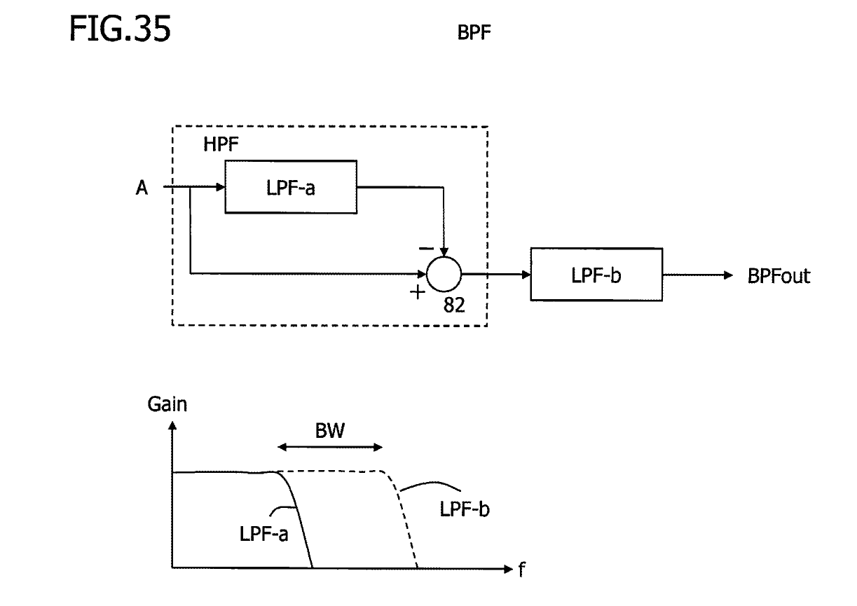

FIG. 35 is a sequence diagram of a computing process by the band pass filter BPF. The computing process by the band pass filter BPF can be realized by performing a computation by the high pass filter HPF and further performing a computation by a low pass filter LPF-b on an output of the high pass filter HPF. In a similar manner to FIG. 34, the computing process by the high pass filter HPF is realized by subtracting an output of the low pass filter LPF-a from the sensor value A. In this case, a cutoff frequency of the low pass filter LPF-a in the high pass filter HPF is set lower than a cutoff frequency of the low pass filter LPF-b in a subsequent stage. In order to do so, for example, the cutoff frequency is lowered by increasing the number of flip-flops n and the cutoff frequency can be raised by reducing the number of flip-flops n of the low pass filter illustrated in FIG. 33. An extraction process by the BPF is also realized with the acceleration sensor output processing program 15.

As described above, by executing the computations described above, the sub microprocessor 14 can realize the functions of an LPF, an HPF, and a BPF.

All examples and conditional language provided herein are intended for the pedagogical purposes of aiding the reader in understanding the invention and the concepts contributed by the inventor to further the art, and are not to be construed as limitations to such specifically recited examples and conditions, nor does the organization of such examples in the specification relate to a showing of the superiority and inferiority of the invention. Although one or more embodiments of the present invention have been described in detail, it should be understood that the various changes, substitutions, and alterations could be made hereto without departing from the spirit and scope of the invention.

What is claimed is:

1. A non-transitory computer-readable storage medium storing therein an acceleration sensor output processing program for causing a computer to execute a process comprising:
generating an outer product of a gravity component vector that is extracted from a sensor value of a triaxial acceleration sensor which is detected during walking by a low pass filter and a swing component vector that is extracted from the sensor value by a high pass filter;
obtaining a first value corresponding to a magnitude of the outer product;
determining an odd-numbered walking period and an even-numbered walking period of the walking; and
generating a magnitude of acceleration in one and the other horizontal directions by inverting a sign of the first value in accordance with a result of the determining.

2. The non-transitory computer-readable storage medium according to claim 1, wherein
the determining includes,
generating an inner product of the gravity component vector and the swing component vector and,
based on an odd-numbered period and an even-numbered period of the inner product, determining the odd-numbered walking period and the even-numbered walking period of the walking.

3. The non-transitory computer-readable storage medium according to claim 1, wherein
the determining includes determining, based on a sign of an inner product of the outer product and an arbitrary vector, the odd-numbered walking period and the even-numbered walking period of the walking.

4. The non-transitory computer-readable storage medium according to claim 1, wherein
the determination includes determining, based on a sign of an inner product with a maximum absolute value among inner products of the outer product and at least three vectors that are not on a same plane, the odd-numbered walking period and the even-numbered walking period of the walking.

5. The non-transitory computer-readable storage medium according to claim 1, wherein
the determination includes,
determining, based on a sign of an inner product of the outer product and an arbitrary vector, the odd-numbered walking period and the even-numbered walking period of the walking, and
generating the arbitrary vector by multiplying the outer product with a sign of the inner product and averaging the outer product.

6. A non-transitory computer-readable storage medium storing therein an acceleration sensor output processing program for causing a computer to execute a process comprising:
generating a first outer product by calculating an outer product of a gravity component vector that is extracted from a sensor value of a triaxial acceleration sensor which is detected during walking by a low pass filter and a front-back swing component vector that is extracted from the sensor value by a first band pass filter;
generating a horizontal reference vector having one horizontal direction and a certain magnitude by multiplying the first outer product by a prescribed sign that changes alternately and averaging the first outer product;
generating the prescribed sign based on a sign of a first inner product of the first outer product and the horizontal reference vector; and
calculating an inner product of a horizontal swing component vector that is extracted using a second band pass filter, whose passing frequency band is lower than that of the first band pass filter, from the sensor value and the horizontal reference vector to generate a first magnitude of acceleration in one and the other horizontal directions.

7. The non-transitory computer-readable storage medium according to claim 6, the process further comprising:
calculating an outer product of the gravity component vector and the horizontal reference vector to generate a front-back reference vector having one of the front-back directions and a predetermined magnitude; and
calculating an inner product of the front-back swing component vector and the front-back reference vector to generate a second magnitude of acceleration in one and the other front-back directions.

8. The non-transitory computer-readable storage medium according to claim 6, the process further comprising:
modifying a sign of the first magnitude based on a sign of a second inner product of a swing component vector that is extracted by a high pass filter from the sensor value and the gravity component vector to generate a modified first magnitude that enables horizontal directions to be distinguished from each other.

9. The non-transitory computer-readable storage medium according to claim 6, the process further comprising:
modifying a sign of the second magnitude based on the sign of the second inner product of a swing component vector that is extracted by a high pass filter from the sensor value and the gravity component vector to generate a modified second magnitude that enables front-back directions to be distinguished from each other.

10. A non-transitory computer-readable storage medium storing therein a gait assessment program for causing a computer to execute a process comprising:
generating a ratio of magnitudes of acceleration in one and the other horizontal directions that are generated by the acceleration sensor output processing according to claim 1; and
performing a gait assessment based on the ratio.

11. A non-transitory computer-readable storage medium storing therein a gait assessment program for causing a computer to execute a process comprising:
generating an accumulated value of magnitudes of acceleration in one and the other horizontal directions that are generated by the acceleration sensor output processing according to claim 1; and
performing a gait assessment based on the accumulated value.

12. A non-transitory computer-readable storage medium storing therein a gait assessment program for causing a computer to execute a process comprising:
generating positional information in one and the other horizontal directions by twice integrating magnitudes of acceleration in one and the other horizontal directions that are generated by the acceleration sensor output processing according to claim 1; and
performing a gait assessment based on the positional information.

13. An acceleration sensor output processing apparatus comprising:
an outer product generator configured to generate an outer product of a gravity component vector that is extracted from a sensor value of a triaxial acceleration sensor which is detected during walking by a low pass filter and a swing component vector that is extracted from the sensor value by a high pass filter;
a first value generator configured to generate a first value corresponding to a magnitude of the outer product;
a determining unit configured to determine an odd-numbered walking period and an even-numbered walking period of the walking; and
a magnitude generator configured to generate the magnitude of acceleration in one and the other horizontal directions by inverting a sign of the first value in accordance with a determination result of the determining unit.

14. The acceleration sensor output processing apparatus according to claim 13, wherein
the determining unit generates an inner product of the gravity component vector and the swing component vector and, based on an odd-numbered period and an even-numbered period of the inner product, determines the odd-numbered walking period and the even-numbered walking period of the walking.

15. The acceleration sensor output processing apparatus according to claim 13, wherein
the determining unit determines, based on a sign of an inner product of the outer product and an arbitrary vector, the odd-numbered walking period and the even-numbered walking period of the walking.

16. The acceleration sensor output processing apparatus according to claim 13, wherein
the determining unit determines, based on a sign of an inner product with a maximum absolute value among inner products of the outer product and at least three vectors that are not on a same plane, the odd-numbered walking period and the even-numbered walking period of the walking.

17. The acceleration sensor output processing apparatus according to claim 13, wherein
the determining unit determines, based on a sign of an inner product of the outer product and an arbitrary vector, the odd-numbered walking period and the even-numbered walking period of the walking, and generates the arbitrary vector by multiplying the outer product with a sign of the inner product and averaging the outer product.

18. An acceleration sensor output processing apparatus comprising:
an outer product generator configured to generate a first outer product by calculating an outer product of a gravity component vector that is extracted from a sensor value of a triaxial acceleration sensor which is detected during walking by a low pass filter and a front-back swing component vector that is extracted from the sensor value by a first band pass filter;
a horizontal reference vector generator configured to generate a horizontal reference vector having one of the horizontal directions and a certain magnitude by multiplying the first outer product by a prescribed sign that changes alternately and averaging the first outer product;
a sign generator configured to generate the prescribed sign based on a sign of a first inner product of the first outer product and the horizontal reference vector; and
a first magnitude generator configured to calculate an inner product of a horizontal swing component vector that is extracted using a second band pass filter, whose passing frequency band is lower than that of the first band pass filter, from the sensor value and the horizontal reference vector to generate a first magnitude of acceleration in one and the other horizontal directions.

19. The acceleration sensor output processing apparatus according to claim 18, further comprising:
a front-back reference vector generator configured to calculate an outer product of the gravity component vector and the horizontal reference vector to generate a front-back reference vector having one of the front-back directions and a predetermined magnitude; and
a second magnitude generator configured to calculate an inner product of the front-back swing component vector and the front-back reference vector to generate a second magnitude f of acceleration in one and the other front-back directions.

20. The acceleration sensor output processing apparatus according to claim 18, further comprising:
a modified first magnitude generator configured to modify a sign of the first magnitude based on a sign of a second inner product of a swing component vector that is extracted by a high pass filter from the sensor value and the gravity component vector to generate a modified first magnitude that enables horizontal directions to be distinguished from each other.

21. The acceleration sensor output processing apparatus according to claim 18, further comprising:
a modified second magnitude generator configured to modifying a sign of the second magnitude based on the sign of the second inner product of a swing component vector that is extracted by a high pass filter from the sensor value and the gravity component vector to generate a modified second magnitude that enables front-back directions to be distinguished from each other.

22. An acceleration sensor output processing method comprising:
generating an outer product of a gravity component vector that is extracted from a sensor value of a triaxial acceleration sensor which is detected during walking by a low pass filter and a swing component vector that is extracted from the sensor value by a high pass filter;
obtaining a first value corresponding to a magnitude of the outer product;
determining an odd-numbered walking period and an even-numbered walking period of the walking; and
generating a magnitude of acceleration in one and the other horizontal directions by inverting a sign of the first value in accordance with a result of the determining.

23. An acceleration sensor output processing method comprising:
generating a first outer product by calculating an outer product of a gravity component vector that is extracted from a sensor value of a triaxial acceleration sensor which is detected during walking by a low pass filter and a front-back swing component vector that is extracted from the sensor value by a first band pass filter;
generating a horizontal reference vector having one horizontal direction and a certain magnitude by multiplying the first outer product by a prescribed sign that changes alternately and averaging the first outer product;
generating the prescribed sign based on a sign of a first inner product of the first outer product and the horizontal reference vector; and
calculating an inner product of a horizontal swing component vector that is extracted using a second band pass filter, whose passing frequency band is lower than that of the first band pass filter, from the sensor value and the horizontal reference vector to generate a first magnitude of acceleration in one and the other horizontal directions.
